(19) United States
(12) Patent Application Publication
(10) Pub. No.: US 2024/0415882 A1
(43) Pub. Date: Dec. 19, 2024

(54) MacrophageexpressingChimeric Antigen Receptor for Binding FAP antigen and its construction method and uses thereof

(71) Applicant: Zhejiang University, Hangzhou City (CN)

(72) Inventors: Hongwei OUYANG, Hangzhou City (CN); Jiajie HU, Hangzhou City (CN); Jiasheng WANG, Hangzhou City (CN)

(21) Appl. No.: 18/467,429

(22) Filed: Sep. 14, 2023

(30) Foreign Application Priority Data
Jun. 13, 2023 (CN) 2023107004731
Jun. 13, 2023 (CN) 2023107005058

(51) Int. Cl. A61K 35/15; C07K 14/725; C12N 15/86

(52) U.S. Cl. CPC A61K 35/15; C07K 14/7051; C12N 15/86; A61K 38/00; C07K 2317/565; C07K 2317/622; C07K 2317/73; C12N 2740/15043

(57) ABSTRACT

A fibroblast activation protein (FAP) targeting chimeric antigen receptor macrophage and its construction method and application. The expression of FAP increases in senescent cells of various senescent diseases. Immune cells, especially macrophages, specifically eliminates senescent cells after expressing chimeric antigen receptor recognizing FAP protein, thereby reducing the pathogenic effect of senescent cells. Macrophages expressing the FAP chimeric antigen receptor can undergo M2-type polarization, which do not produce inflammatory factors to deteriorate age related diseases, have good infiltration ability to tissues with dense matrix, do not induce graft-versus-host disease and have high recognition efficiency of senescent cells and good phagocytic effect. This provides a new idea for preparing reagents for eliminating senescent cells, which have wide potential in practical application.

### CROSS-REFERENCE TO RELATED APPLICATIONS

This application claims priority to Chinese Patent Application No.202310700505.8, filed on 13 Jun. 2023 and Chinese Patent Application No.202310700473.1, filed on 13 Jun. 2023, and the specification, abstract, accompanying drawings, and claims of this application are made a part of this application.

### REFERENCE TO AN ELECTRONIC SEQUENCE LISTING

The content of the electronic sequence listing (MacrophageexpressingChimeric_Antigen_Receptor.xml; Size: 28,310 bytes; Date of Creation: Sep. 14, 2023) is herein incorporated by reference in its entirety.

### TECHNICAL FIELD

The invention relates to the field of biomedicine, a chimeric antigen receptor targeting FAP is specifically involved.

### BACKGROUND TECHNOLOGY

The following background technical introduction is only an introduction of some background knowledge and does not constitute any limitation of the invention.

Senolytics (drugs that selectively eliminate senescent cells) have attracted a lot of attention since they are reported. Since 30-70% of senescent cells with pro-apoptotic, tissue-destructive Senescence Associated Secretory Phenotype (SASP) are themselves resistant to apoptosis. Analysis of proteomic and transcriptomic data demonstrates the upregulation of one or more Senescent Cell Anti-apoptotic Pathways (SCAPs) exist in senescent cells. The SCAPs protect senescent cells from apoptosis and instantaneous blocking of the SCAPs can lead to apoptosis of senescent cells, and non-senescent cells can still survive. However, due to the large heterogeneity of senescent cells, SCAPs are redundant in certain types of senescent cells; Senolytics targeting a single SCAP cannot effectively clear all types of senescent cells. In addition, some Senolytics, such as BCL-2 inhibitors ABT-263, A1331852, or A1155463, may cause thrombocytopenia and neutropenia. Therefore, although Senolytics alleviate a variety of age-related disorders in a variety of preclinical mouse models, some studies in the Senolytics clinical trial are terminated early due to poor efficacy (NCT04349956).

Another way to reduce senescent cell accumulation and alleviate related pathological conditions is directing the immune clearance of senescent cells. This is because some cell surface proteins (antigens) tend to be expressed higher in senescent cells than in other cells, such as non-senescent cells, which led to the development of Chimeric Antigen Receptor (CAR)-T cells that can target these cell surface markers. In 2020, C. Amor, J reported for the first time that CAR-T cells targeting Urokinase Plasminogen Activator Surface Receptor (PLAUR) can specifically eliminate PLAUR positive cells, which targetly kill senescent cells both in vivo and in vitro, attenuate senescence related liver fibrosis and prolong the lifespan of mice with lung adenocarcinoma. However, CAR-T cell therapy also faces the drawbacks of the expensive cost, the inadequate trafficking or penetration in solid tissues, the possibility of triggering inflammatory cytokine release syndrome (CRS) and the transplant rejection requiring long-term risky immunosuppressive therapy.

Single Senolytics is less effective and may cause toxic and side effects such as thrombocytopenia and neutropenia, while conventional CAR-T cells therapy is expensive, insufficient to infiltrate into solid tissue, and may cause inflammatory factor storms and graft-versus-host disease.

Therefore, it is urgent to improve the traditional technology and identify new senescent cell surface markers, which can be used to specifically eliminate the senescent cells and attenuate age-related diseases.

### SUMMAR OF THE INVENTION

To overcome the defects of the traditional technology, the invention provides the use of FAP protein in preparing reagents to inhibit or eliminate senescent cells or providing an agent that can bind the FAP so as to inhibit or eliminate senescent cells in the individual.

The invention finds that the Fibroblast Activation Protein Alpha (FAP, Ensembl: ENSG00000078098, UniProtKB/Swiss-Prot: Q12884) Surface proteins are commonly upregulated in a wide range of in vitro and in vivo mammalian aging models. The amino acid sequence of the FAP protein (antigen) is shown as SEQ ID: 1, the nucleic acid encoding the FAP protein has the sequence as SEQ ID: 2, and the DNA expressing FAP is the gene encoding NG_027991.1.

Upregulation of FAP in response to all tested aging triggers: replication-induced senescence, drug-induced senescence (e.g. Doxorubicin, DOX) and oncogene-induced senescence. The upregulation of the FAP protein is present in most senescent cells, for example in some age-related diseases such as pulmonary fibrosis, atherosclerosis, Alzheimer's disease, diabetes, liver cirrhosis, chronic kidney disease, osteoporosis or osteoarthritis. Upregulation of FAP is also found in senescent cells in tumor subjects receiving senescence-inducing therapies, such as chemotherapy drugs.

Therefore, the invention demonstrates FAP is associated with senescent cells, and the upregulation of FAP indicates the increase or accumulation of senescent cells, which may start from the diseased tissues or organs then to the systemic accumulation.

All methods that can inhibit the expression of FAP protein can be used to eliminate or inhibit senescent cells.

For example, a monoclonal antibody, an antibody fragment or a peptide that binds a FAP protein can inhibit senescent cells. Carriers or immune cells expressing anti-FAP antibodies, antibody fragments or peptides can also specifically kill or eliminate senescent cells through binding the FAP protein.

It can also be some nucleic acid drugs, targeted delivery to senescent cells through vectors, thereby inhibiting the expression of FAP protein, RNA transcription or translation or FAP protein formation process.

Or it can be some chemical compound that binds to FAP proteins to eliminate or inhibit senescent cells. Monoclonal antibodies, antibody fragments, peptides or chemical agents expressed in vitro or in vivo that can bind to any position of the FAP amino acid sequence can inhibit FAP activity. In addition, FAP protein can be reduced or not expressed by gene knockout, gene editing, and gene silencing.

Furthermore, engineered immune cells can be used to effectively eliminate senescent cells that express FAP (Gene ID: 2191). This engineered immune cell can specifically eliminate FAP positive senescence cells without causing any undesirable toxicity/side effects to the host. Any engineered immune cell can be used to express antibodies, antibody fragments or peptides against FAP. Any reagent that can bind FAP protein can inhibit the activity of FAP and thereby inhibit or eliminate FAP positive senescent cells.

It is understood that the above amino acid fragments binding FAP antigens or nucleic acid fragments expressing amino acids are not necessarily obtained by engineered immune cells, but can also be obtained by any other methods, such as in vitro expression like yeast expression, delivered to senescent cells containing FAP by vector for expression or genetic engineering approach to edit, mutate or silence FAP gene. Engineered immune cells is one option.

Therefore, the invention provides a method for inhibiting or eliminating senescent cells comprising applying an agent to the individual, wherein the agent can bind an FAP protein.

In some embodiments, the reagents include the CARs targeting FAP, which comprises a FAP binding region. In some embodiments, the CAR structure includes extracellular domains, transmembrane domains and intracellular domains. In some embodiments, the extracellular domain includes a region that specifically bind FAP as well as spacer regions.

Further, the senescent cell eliminating agents also include cells engineered by CARs targeting FAP proteins. The cells include any one or more of monocytes, macrophages, or dendritic cells.

Further, the senescent cell eliminating agent is cells. The cell is macrophage. Macrophages have the unique tissue infiltration capacity to penetrate solid tumors, while other immune cells (such as T cells) are rejected or inactivated. Therefore, macrophages containing FAP specific CARs can be used to eliminate senescent cells within solid tumors. In addition, macrophages do not induce graft-versus-host disease, raising their prospects as a source of off-the-shelf cells. Due to their natural phagocytosis and unique tissue penetration, engineered macrophages have an innate advantage in infiltrating and eliminating senescent cells in tissues with dense matrix, such as articular cartilage or synovium.

Further, the senescent cells induction modalities include any one or more of replication-induced senescence, drug-induced senescence and oncogene-induced senescence. The senescent cell has FAP protein on its surface, the amino acid sequence of the FAP is as shown in SEQ ID: 1, and the nucleic acid encoding the FAP protein has a sequence as shown in SEQ ID: 2.

Further, the drug-induced senescent cells include senescent cells induced by MEK inhibitors and/or Cdk4/6 inhibitors. The MEK inhibitors include trametinib and/or doxorubicin. The Cdk4/6 inhibitor includes palbociclib. The oncogenes include any one or more of HRASG12D, NRASG12D, and D38A.

On the other hand, the invention provides the use of FAP protein in the preparation of reagents for the treatment of diseases caused by senescent cells.

Further, the diseases caused by senescent cells include any one or more of cardiac fibrosis, pulmonary fibrosis, atherosclerosis, Alzheimer's disease, diabetes, liver cirrhosis, chronic kidney disease, aging, osteoporosis or osteoarthritis.

On the other hand, the invention provides the use of FAP proteins for preparing reagents for predicting or detecting senescent cells. FAP is associated with senescent cells, the expression of FAP is increased in senescent cells, and the expression level of FAP can be used as a standard to judge whether the cells are senescent. FAP can be used as a surface marker of senescent cells in immunotherapy of osteoarthritis. The detection method of the senescent cell surface markers is as follows: qPCR, immunofluorescence and flow cytometry for relative quantification, and the senescent cells can be judged if the FAP shows statistically significant difference compared with the control.

On the other hand, the invention provides a CAR for the preparation of reagents to eliminate senescent cells, treat diseases caused by senescent cells or osteoarthritis. The CAR contains an antibody fragment that binds the FAP.

On the other hand, the invention provides an immunoengineered cell for the preparation of the elimination of senescent cells or the treatment of diseases caused by senescent cells or osteoarthritis. The immunoengineered cell contains the CAR. The CAR contains an antibody fragment that binds the FAP.

Further, the immunoengineered cells are any one or more of macrophages, monocytes, or dendritic cells. Macrophages contain FAP-specific CARs can selectively eliminate senescent cells expressing FAP, exert their phagocytic activity, and achieve the purpose of eliminating senescent cells without affecting normal proliferating cells or causing any undesirable toxicity/side effects to the host.

On the other hand, the invention provides a CAR comprising an extracellular domain, a transmembrane domain and an intracellular domain. Extracellular domains include an antigen-binding domains that binds the targeting FAP.

Further, the extracellular domain of the CAR includes a targeting FAP antigen-binding domains. The targeting FAP antigen-binding domain is an antibody fragment that can bind the FAP antigen. The FAP has an amino acid sequence as shown in SEQ ID: 1, and the nucleic acid encoding the FAP protein has a sequence as shown in SEQ ID: 2.

Further, the extracellular domain of the CAR contains, but is not limited to, scFv, Fab, scFab, or scIgG antibody fragments for recognizing and binding FAP-specific/associated antigens.

In some embodiments, the antibody fragment includes a heavy chain and/or a light chain. The heavy chain includes any one or more of VHCDR1, VHCDR2, VHCDR3, the light chain includes any one or more of VLCDR1, VLCDR2, VLCDR3. The VHCDR1, VHCDR2, VHCDR3, VLCDR1, VLCDR2, and VLCDR3 have amino acid sequences as shown in SEQ ID: 4˜9, respectively, or a sequence that is 60%, 70%, 80%, 85%, 90%, or more than 95% homologous to any one or more of the amino acid sequences shown in SEQ ID: 4˜9.

In some embodiments, the antibody fragment is scFv. The scFv contains a heavy chain and/or a light chain. The amino acid sequence of the heavy chain is shown as SEQ ID NO: 10, the amino acid sequence of the light chain is shown as SEQ ID: 11, or sequences that are homologous to 60%, 70%, 80%, 85%, 90%, or more than 95% of the amino acid sequences shown in SEQ ID: 10 and/or SEQ ID: 11.

Further, the scFv has a nucleotide sequence as indicated by SEQ ID: 12 and/or SEQ ID: 13, or a sequence that is 60%, 70%, 80%, 85%, 90%, or more than 95% homologous to a nucleotide sequence as indicated by SEQ ID: 12 and/or SEQ ID: 13.

In some embodiments, the co-stimulatory domain can enhance the receptor signal, and the co-stimulatory domain includes, but not limited to, one or more domains of the 4-1BB (CD137), CD80, CD86, FcεRI common gamma subunit (i.e. FcsRIγ), and CD28 intracellular domains. The 4-1BB has an amino acid sequence as shown in SEQ ID: 24, or a sequence that is 60%, 70%, 80%, 85%, 90%, or more than 95% homologous to the amino acid sequence as shown in SEQ ID: 24. The 4-1BB has a nucleic acid sequence as shown in SEQ ID: 3, or a sequence that is 60%, 70%, 80%, 85%, 90%, or more than 95% homologous to a nucleic acid sequence as shown in SEQ ID: 3.

In some embodiments, CD28 intracellular domain is preferred. When the CAR is engineered in macrophages, the use of CD28 intracellular domain as a co-stimulatory domain has the following advantages: 1. CD28 can promote the anti-inflammatory M2 polarization of macrophages, which can be applied to the diseases that need to avoid or inhibit inflammation. 2. CD28 intracellular domain can make macrophages have a higher phagocytosis effect; 3. CD28 intracellular domain allows macrophages expressing CARs have a lower response threshold to FAP antigen density on target cells, especially for the phagocytosis of senescent chondrocytes with low FAP expression, in which CD28 intracellular domain has more obvious advantages than other co-stimulatory domains. The amino acid sequence of the CD28 intracellular domain is such as the amino acid sequence shown in SEQ ID: 14, or a sequence that is 60%, 70%, 80%, 85%, 90%, or more than 95% homologous to the amino acid sequence shown in SEQ ID: 14. The CD28 intracellular domain has a nucleic acid sequence as shown in SEQ ID: 15, or a sequence that is 60%, 70%, 80%, 85%, 90%, or more than 95% homologous to a nucleic acid sequence as shown in SEQ ID: 15.

Macrophages with FcεRI common γ subunit or CD28 co-stimulating domain have higher phagocytosis effect. In some Embodiments, when the senescent cells are human senescent chondrocytes with low FAP expression level, CAR-M containing CD28 intracellular domain has a higher phagocytosis rate of 38.75% against FAP-positive target cells. When senescent cells are senescent fibroblasts, the phagocytic effect of macrophages is increased to 41.5% and 37.0% by FcεRIγ and CD28 intracellular domains, respectively.

It is understood that any peptide fragment that can stimulate an enhanced receptor signal can be included in the co-stimulatory domain of the CARs provided by the invention. Co-stimulatory molecules include, but are not limited to, TCR, CD3 zeta, CD3 gamma, CD3 delta, CD3 epsilon, CD86, common FcR gamma, FcR beta (Fc Epsilon Rib), CD79a, CD79b, FcgammaRlla, DAPIO, DAP 12, CD27, CD28, 4-1BB (CD137), OX40, CD30, CD40, PD-1, ICOS, Lymphocyte Function Associated Antigen-1 (LFA-1), CD2, CD7, LIG HT, KG2C, B7-H3, ligands that can specifically bind to CD83, CDS, ICAM-1, GITR, BAFFR, HVEM (LIGHTR), SLAMF7, Kp80 (KLRF1), CD127, CD160, CD19, CD4, CD8alpha, CD8beta, IL2R,IL2R gamma, IL7R alpha, ITGA4, VLAI, CD49a, ITGA4, IA4, CD49D, ITGA6, VLA-6, CD49f, ITGAD, CDlld, ITGAE, CD103, ITGAL, CD11a, LFA-1, ITGAM, CDllb, ITGAX, CD11c, ITGB1, CD29, ITGB2, CD18, LFA-1, ITGB7, TFR2, TRANCE/RAKL, DNAM1 (CD226), SLAMF4 (CD244, 2B4), CD84, CD96, CEACAM1, CRTAM, Ly9 (CD229), CD160 (BY55), PSGLI, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, Kp44, Kp30, Kp46, KG2D. The other co-stimulatory molecules described in the invention, any derivative, variant or fragment thereof, any synthetic sequence of a co-stimulatory molecule with features that enhance the stimulus signal has the same functional capability, and any combination thereof.

In some embodiments, the intracellular activation region includes, but not limited to, one or more of FcεRIγ, CD3ζ. Intracellular activated regions can transduct signals. Preferably, the intracellular active region is CD3ζ, and the CD3ζ, has an amino acid sequence as shown in SEQ ID: 16, or a sequence that is 60%, 70%, 80%, 85%, 90%, or more than 95% homologous to the amino acid sequence as shown in SEQ ID: 16. The CD3ζ has a nucleic acid sequence as shown in SEQ ID: 17, or a sequence that is 60%, 70%, 80%, 85%, 90%, or more than 95% homologous to a nucleic acid sequence as shown in SEQ ID: 17. FcεRIγ contains an immunoreceptor tyrosine-based activation motif (ITAM), which is an important signal transduction region mediating phagocytosis activity of macrophages. CD3ζ contains three ITAMs, which are similar in structure, distribution and function to FcεRIγ. The more of ITAMs, the stronger downstream signal and the stronger response of immune cells. The FcsRIγ has an amino acid sequence as shown in SEQ ID: 22, or a sequence that is 60%, 70%, 80%, 85%, 90%, or more than 95% homologous to the amino acid sequence as shown in SEQ ID: 22.The FcsRIγ has a nucleic acid sequence as shown in SEQ ID: 23, or a sequence that is 60%, 70%, 80%, 85%, 90%, or more than 95% homologous to a nucleic acid sequence as shown in SEQ ID: 23.

In some embodiments, the transmembrane domain contains CD28 or CD8.The CD28 and CD8 have, respectively, an amino acid sequence as shown in SEQ ID: 18˜19, or a sequence that is 60%, 70%, 80%, 85%, 90%, or more than 95% homologous to the amino acid sequence as shown in SEQ ID: 18˜19. The CD28 and CD8 have nucleic acid sequences as shown in SEQ ID: 20˜21, respectively, or sequences that are 60%, 70%, 80%, 85%, 90%, or more than 95% homologous to nucleic acid sequences as shown in SEQ ID: 20˜21.

In some embodiments, the extracellular domain of the CAR also includes a spacer region connecting the FAP targeting antigen-binding domain and the transmembrane domain.

In some embodiments, the spacer region can be flexible enough to allow the antigen-binding domain to be oriented in different directions, which can facilitate antigen recognition while maintaining CAR activation activity. In some Embodiments, the adoption of CD8 in the transmembrane domain of the CAR will have an a-hinge of CD8 expressed in the spacer region, which facilitates the response of the CAR to the FAP antigen density on the target cell. CAR macrophages are allowed to recognize senescent chondrocytes with low FAP expression (FAP expression is upregulated 20-fold relative to non-senescent cells).

In some non-restrictive Embodiments, spacer regions include, but not limited to, a part of CD28, a part of CD8, a part of immunoglobulin CH2CH3, a part of CD3, and any one or more of the hinge regions of IgGl. The CD28 and CD8 have an amino acid sequence as shown in SEQ ID: 18˜19, respectively, or sequences that is 60%, 70%, 80%, 85%, 90%, or more than 95% homologous to the amino acid sequence as shown in SEQ ID: 18˜19.The CD28 and CD8 have nucleic acid sequences as shown in SEQ ID: 20˜21, respectively, or sequences that is 60%, 70%, 80%, 85%, 90%, or more than 95% homologous to the nucleic acid sequences as shown in SEQ ID: 20˜21, or synthetic interval sequences. Any such variant is homologous to, or synthetically spaced from at least 80%, 85%, 90%, or 95%. In some non-restrictive Embodiments, the length of the spacer region may be between approximately 1-50 (e.g., 5-25, 10-30, or 30-50) amino acids.

On the other hand, the invention provides a modified cell containing a CAR. Cells express CARs provided by the invention. The cells are immune cells. In some embodiments, the cell is one or more of monocytes, macrophages, or dendritic cells.

In some Embodiments, any number of monocytes, macrophages, dendritic cells, or progenitor cell lines available in the field may be used. These cells do not necessarily come from the subjects. In some Embodiments, cells may be obtained from blood units collected by the subject using any technique known to those skilled in the art, such as Ficoll. In one Embodiment, cells or groups of cells containing monocytes, macrophages, or dendritic cells are cultured for amplification. In another Embodiment, cells or populations of cells containing progenitor cells are cultured for differentiation and expansion of monocytes, macrophages, or dendritic cells. In unrestricted alternatives, monocytes or macrophages derived from pluripotent stem cells (such as embryonic stem cells) can also be used as alternatives.

The invention includes the expansion of populations of monocytes, macrophages or dendritic cells containing the CAR provided by the invention.

Further, the cells express CARs by transfecting cells with vectors including plasmid vectors, viral vectors, retrotransposons (e.g. piggyback, Sleeping Beauty), site-inserted vectors (e.g. CRISPR, Zincfinger nuclease, TALEN), or suicide expression vectors, or other vectors known in the field.

In one Embodiment, preferably, the vector is a lentiviral vector. Transfection of macrophages with lentivirus as a vector can avoid the generation of pro-inflammatory M1 polarization, which is more beneficial to promote the generation of anti-inflammatory M2 polarization. Lentiviral vector avoids the current use of Ad5f35 adenovirus vectors for macrophage M1 polarization. When macrophages generate pro-inflammatory M1 polarization, they are not suitable for senescent cell therapy due to the production of pro-inflammatory cytokines such as interleukin-1β (IL-1β), IL-6, IL-12, IL-23, and TNF-α. Only the anti-inflammatory M2 type polarized macrophages are suitable for tissue regeneration therapy in degenerative diseases. Preferably, when the co-stimulatory domain of the CAR provided by the invention expressed by macrophages has CD28 intracellular domain, lentiviral vector can stably promote the M2-type polarization of macrophages.

The current method to promote M2-polarized macrophages is to transfect CARs while further transfecting signaling factors to inhibit the pathway that generates M1-polarized macrophages, such as TNF-α/NF-KappaB pathway and TLR/MyD88 pathway to realize M2-polarized macrophages. The regulation method of M2-type polarized macrophages provided by the invention only needs to express CAR without expressing any other signal factors, which is more efficient and stable than the prior art.

In some embodiments, the vector used is a lentivirus, the use of spinfection increased the expression rate of CARs by 4.7 times to 57%.

When FAP-specific CAR macrophages are applied to the treatment of mice or human osteoarthritis models, they can effectively eliminate FAP-positive senescent cells and generate therapeutic effects, such as a liminating senescent chondrocyte. In some Embodiment, the FAP-specific CAR macrophage provided by the invention is applied to the treatment of the mouse model of traumatic osteoarthritis, which can not only effectively remove senescent cells, but also reduce synovial inflammation, and restore the motor dysfunction of the mouse.

On the other hand, the invention provides a preparation method of modified cells containing CARs. Macrophages are transfected with lentivirus as a vector and spinfection is performed. In some Embodiments, the parameters for spinfection are: 800× g for 20 min.

On the other hand, the invention provides a method for inhibiting or eliminating senescent cells. The method inhibits or eliminates senescent cells by applying agents that inhibit or eliminate senescent cells to an individual, including an agent for targeting FAP proteins.

Further, the FAP has an amino acid sequence as shown in SEQID: 1. The nucleic acid encoding the FAP protein has a sequence as shown in SEQID: 2.

Further, the reagents targeting the FAP protein include CARs as described above.

Further, the reagents targeting the FAP protein also include cells. The cells are modified by a CAR targeting the FAP protein. The cells include any one or more of monocytes, macrophages, or dendritic cells.

Further, the cells are macrophages.

Further, the senescent cells include senescent cells induced by any one or more of cell replication, drugs, or oncogenes.

Further, the drug-induced senescent cells include senescent cells induced by MEK inhibitors and/or Cdk4/6 inhibitors. The MEK inhibitors include trametinib and/or doxorubicin. The Cdk4/6 inhibitor includes palbociclib. The oncogenes include any one or more of HRASG12D, NRASG12D and D38A.

On the other hand, the invention provides a method for the preparation of reagents for the treatment of diseases caused by senescent cells. The method treats diseases caused by senescent cells by applying agents that inhibit or eliminate senescent cells to an individual. The agents that inhibit or eliminate senescent cells include CARs as described above.

Further, the agent for inhibiting or eliminating senescent cells also includes cells. The cells are modified by CARs targeting FAP proteins. The cells include any one or more of monocytes, macrophages, or dendritic cells.

Further, the diseases caused by senescent cells include any one or more of cardiac fibrosis, pulmonary fibrosis, atherosclerosis, Alzheimer's disease, diabetes, liver cirrhosis, chronic kidney disease, aging, osteoporosis or osteoarthritis.

On the other hand, the invention provides a method for regulating the polarization of macrophages. The macrophages are transfected with lentiviral vectors to express CARs. The CAR contains a co-stimulatory domain of the CD28 intracellular domain that causes macrophages to produce anti-inflammatory M2-type polarization.

Further, the co-stimulatory domain containing the CD28 intracellular domain is located in the intracellular domain of the CAR. The extracellular domain of the CAR also includes the targeting antigen-binding domain. The CD28 intracellular domain has an amino acid sequence as shown in SEQ ID: 14, or a sequence that is 60%, 70%, 80%, 85%, 90%, or more than 95% homologous to the amino acid sequence as shown in SEQ ID: 14. The CD28 intracellular domain has a nucleic acid sequence as shown in SEQ ID: 15, or a sequence that is 60%, 70%, 80%, 85%, 90%, or more than 95% homologous to a nucleic acid sequence as shown in SEQ ID: 15.

In some embodiments, the extracellular domains include antigen-binding domains that specifically bind cell surface proteins, as well as spacer regions.

Further, the antigen-binding domain can directional bind target cells whose antigen density of the target protein reaches a response threshold, without binding to other cells. The antigen-binding domain is an antibody fragment that can be designed according to the target protein. The target protein includes any cell surface protein. The target cells can be cancer cells, senescent cells, or any other cells.

In some embodiments, the target protein is any protein on the surface of senescent cells, such as the FAP protein in the invention, and also includes a specific protein on the cell surface of senescent cells that has been discovered, such as the urokinase plasminogen activator receptor (uPAR) and Glycoprotein Nmb (GPNMB). Of course, it can also be used for other proteins that have not been discovered on the surface of senescent cells, and these proteins, once newly discovered, can be used in the CAR structure of the invention.

In some optional embodiments, the target protein includes, but not limited to, any of the following antigenic compositions: PLAUR, GPNMB, mesothelin, CD1, CD1a, CD2, CD3, CD4, CD5, CD8, CD11A, CD14, CD15, CD16, CD18, CD19, CD21, CD20, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD126, CD132, CD133, CD138, CD147, CD154, CDC27, GD2, CCL19, CCL21, CDK-4/m, CDKN2A, CTLA4, CXCR4, CXCR7, CXCL12, HER2, CAIX, CD171, LMP1, EGFR, Muc1, GPC3, EphA2, EpCAM, MG7, CSR, ART-4.

On the other hand, the invention provides the use of lentivirus as a carrier to transfect macrophages to express CARs containing CD28 intracellular co-stimulatory domains in regulating macrophages to M2 type polarization.

In summary, the beneficial effects of the invention are as follows:

The invention discovers the relationship between FAP and senescent cells, thereby providing the application of FAP protein in preparing reagents to inhibit or eliminate senescent cells, providing a new idea for the scene requiring the elimination of senescent cells in the design of anti-aging drugs and development of beauty ingredients, etc., and also providing a new use of FAP as a marker of senescent cells.

The CAR specifically targeting FAP and the FAP-specific CAR macrophage provided by the invention can selectively eliminate FAP positive senescent cells without affecting normal proliferating cells, can effectively be applied to the treatment of osteoarthritis to eliminate senescent chondrocytes, and can reduce synovial inflammation at the same time. The cells could also be used to prevent aging.

The immunoengineered cells containing the FAP-specific CAR provided by the invention have the following advantages: 1. Unique tissue infiltration ability, can penetrate solid tumors, tissues with dense matrix such as articular cartilage or synovial membrane; 2. Macrophages do not induce graft-versus-host disease and have higher immune safety than CAR-T cells. Therefore, the use of macrophages as immunoengineered cells is more beneficial to remove senescent cells in tissues with dense matrix or solid tumor.

The CAR molecular signal domain of the FAP-specific CAR macrophage provided by the invention is optimized, and the co-stimulating domain is FcεRI common γ subunit or CD28 intracellular domain, which can improve the phagocytic effect of macrophages.

The polarization phenotype of the FAP-specific CAR macrophage provided by the invention is the anti-inflammatory M2 type, which avoids the traditional M1 polarization after the activation of macrophages, and helps to avoid inflammation in the clearance of senescent cells.

The invention first proposes a stable CAR macrophage M2 type polarization method as follows: Lentivirus is used as a vector to transfect macrophages, and the co-stimulatory domain of the CAR is CD28 intracellular domain. This method could stably obtain M2 macrophages, without the necessary to construct a pathway to inhibit M1-type polarization (such as TNF-α/NF-KappaB pathway and TLR/MyD88 pathway). Therefore, it can be used and not limited to the treatment of diseases that need anti-inflammatory effects to remove senescent cells. The advantage is that it will not induce graft-versus-host disease and will not produce inflammatory factor storms.

The selection method of CAR sequence, the adjustment of spacer region of extracellular domain, transmembrane domain and co-stimulatory domain of intracellular domain provided by the invention make the response threshold of FAP specific CAR macrophages to FAP antigen density on the surface of senescent cells reach the FAP expression level of senescent chondrocytes and senescent fibroblasts, making the FAP specific CAR macrophages safer and more effective.

### DESCRIPTION OF THE DRAWINGS

In order to more clearly state the technical scheme in the Embodiment of the invention or the prior art, the following is a brief introduction of the figures required to be used in the description of the Embodiment or the prior art. It is obvious that the figures described below are merely Embodiments of the invention, and that other figures may be obtained from them without creative effort by the general skilled personnel in the field.

FIG. 1: Illustration of the proposed chimeric antigen receptor (CAR) macrophage therapeutic approach.

FIG. 2: A conceptual graph of a CAR is shown. The diagram shows, from left to right, extracellular domains (extracellular domains in the figure), transmembrane domains (transmembrane domains in the figure), and intracellular domains (intracellular domains in the figure). The extracellular domain contains the antigen-binding domain with targeting function (the targeted fragment in the figure), and in some embodiments also contains the spacer region (the hinge region in the figure) that connects the antigen-binding domain with the transmembrane domain. The intracellular domain contains the co-stimulatory domain and the intracellular activation domain (the intracellular activation signal in the figure).

FIG. 3: Corresponding to the Embodiment 1. Identification of novel markers of senescent cells through single-cell data. FIG. 3A shows UMAP dimensionality reduction in chondrocytes from OA patients, with significant heterogeneity in the expression of senescent cell signature genes and stroma-degrading genes. FIG. 3B shows in contrast to classical senescent cell markers CDKNIA and PLAUR, FAP is specifically expressed in senescent cell subsets that promote matrix degradation (identity=1).

FIG. 4: Corresponding Embodiment 1. The expression of FAP is up-regulated in senescent cells. FAP expression is significantly increased in primary mouse fibroblasts (FIG. 4C-D) and chondrocytes (FIG. 4A-B) after DOX-induced cell senescence.

FIG. 5: Corresponding to Embodiment 1. FAP is expressed on the surface of senescent cells. Flow cytometry shows FAP is highly expressed on the cell surface of mouse and human senescent chondrocytes (FIG. 5A-B).

FIG. 6.1-6.3: Corresponding to Embodiment 1. Feasibility of immunotherapy of FAP as a senescent cell surface marker for OA. The number of FAP-positive and SA-β-gal positive cells in mouse joint tissues (articular cartilage and synovial tissue) is significantly increased in both Destabilization of the Medial Meniscus (DMM)-induced post-traumatic osteoarthritis (FIG. 6.1B-C) and age-related osteoarthritis (FIG. 6.1A). but is hardly detected in sham joints. Similar to the results in mice, FAP is up-regulated in articular cartilage (FIG. 6.2D) and synovial tissue (FIG. 6.2E) in clinical OA patients, while FAP expression is lower in normal human joints. In addition, protein expression levels of FAP are significantly increased in both heart and liver tissues of aged mice. Together, these results demonstrate that FAP may be an ideal candidate target for the senolytic CAR strategy in OA immunotherapy.

FIG. 7: Corresponding to Embodiment 10. CAR expression efficiency in primary macrophages. The CAR consisted of an anti-mouse FAP (m.FAP) single chain fragment variable (scFv) linked to the intracellular 4-1BB co-stimulation and CD3ζ signaling domain (FIG. 7A). Spinfection can significantly improve the expression efficiency of CAR molecules (FIG. 7B).

FIG. 8.1-8.3: Corresponding to Embodiment 6. Phagocytosis efficiency mediated by CAR molecules with different intracellular structures. FIG. 8.1A is a series of schematic diagrams showing specific examples of CAR constructs, and FIG. 8.2B shows that CAR-M containing the intracellular segment of CD28 or the common γ subunit of FcεRI has a higher phagocytosis rate of FAP-positive target cells.

FIG. 9: Corresponding to Embodiment 4 and Embodiment 5. Preparation of FAP-specific CAR macrophages and evaluation of CAR-mediated senolytic activity. The FAP-specific CAR includes anti-mouse FAP (m.FAP) scFV linked to intracellular CD28 co-stimulation and CD3ζ signaling domain. (FIG. 9A). To evaluate antigen-specific phagocytosis potential of FAP-specific CAR-Macs towards the target antigen, we introduced CAR into J774A.1 murine Macs, and HEK293T cells overexpressing FAP are used as target cells. FAP-specific CAR-Macs had a high phagocytosis rate on FAP-positive target cells, whereas vector (containing GFP but no signaling domains) transduced Macs did not show such selective phagocytosis (FIG. 9B). In order to improve the transfection efficiency of macrophages, spinfection is used to introduce CAR into primary bone marrow macrophages of mice, and macrophages effectively expressed CAR after spinfection (FIG. 9C). To evaluate the senolytic activity of FAP-specific CAR-Macs, CAR-Macs derived from BMDMs are then co-cultured with DOX treated mouse primary chondrocytes. High-content cell analyzer (FIG. 9D) and confocal microscope (FIG. 9E) showed CAR rather than vector transduced BMDMs exhibited an enhanced senescent chondrocytes phagocytic activity. In addition, CAR-Macs exhibited cytotoxicity towards senescent chondrocytes in a dose- and time-dependent manner (FIG. 9F, G). Together, these data demonstrated that FAP-targeting CAR-Macs can specific elimination of senescent chondrocytes and direct senolytic activity.

FIG. 10.1-10.3: Corresponding to Embodiment 7. Evaluation of the polarization phenotype of the FAP-specific CAR-M.FAP-specific CAR-M shows an elongated shape with filamentous cytoskeleton staining, which closely similar to those of M2 (anti-inflammatory) phenotype (FIG. 10.1A). To further characterize the phenotype of FAP CAR-Macs, bulk-RNA sequencing is performed. The transcriptomes of CAR transduced, vector transduced, classically activated M1 or alternatively activated M2 and untreated BMDMs are processed by nonbiased principal component analysis (PCA), CAR-Macs clustered toward alternatively activated M2 state and away from classically activated M1 (FIG. 10.1B). Unbiased hierarchical clustering heatmap also revealed CAR-Macs are similar to M2 but significantly distinct from M1 (FIG. 10.1C). Comparing the inflammatory associated differences between CAR transduced and activated Macs showed CAR-Macs had no significant enrichment of pro-inflammatory path embodiments (FIG. 10.2D). Furthermore, CAR-Macs also positively mediated phagocytosis and antigen processing and presentation associated path embodiments (FIG. 10.3E, F). All these results demonstrated that FAP-specific CAR transduced Macs exhibited an anti-inflammatory like phenotype with enhanced phagocytic activity and antigen presentation.

FIG. 11.1-11.4: Corresponding to Embodiment 9. The therapeutic effects of FAP-specific CAR-M on post-traumatic OA in adult mice. Destabilization of the Medial Meniscus (DMM) surgery is first performed in 10-week-old p16-3MR transgenic mice to allow non-invasive monitoring of senescent cells. two doses of intra-articular (IA) injections of CAR-Macs are administered at 2 and 6 weeks after DMM surgery (FIG. 11.1A). CAR-M treatment significantly lowered the luminescence signal in the joint region of DMM mice, indicating effective senescent cells removal in the joint (FIG. 11.1B). Histological assessment showed CAR-Macs significantly reduced cartilage degeneration and synovial inflammation in OA mice compared with vector-transduced Macs or PBS (FIG. 11.2C-E). Immunostaining is performed for FAP, p16INK4a, and high-mobility-group box 1 (HMGB1) to identify senescent cells. In cartilage, the proportion of FAP- and p16INK4a-positive cells after DMM are significantly higher than those in the sham, while HMGB1 showed nuclear accumulation in the sham and loss after DMM. Notably, CAR-Macs not only significantly reduced the number of p16INK4a-positive cells and restored nuclear staining of HMGB1, but also reduced the number of collagen X-positive hypertrophic chondrocytes and increased the containing of collagen II in cartilage matrix in OA mice (FIG. 11.3F, G). Furthermore, Motor function assessed by Rotarod test revealed that CAR-Macs restored motor dysfunction associated with DMM surgery (FIG. 11.4H). Collectively, these results suggested that FAP-specific CAR-M could be used as therapeutic agents in post-traumatic OA.

FIG. 12.1-12.3: Corresponding to Embodiment 8. Clearance of senescence cells by FAP-specific CAR-M in human joint explants.to evaluate the clinical potential of senolytic CAR-Macs in OA, we collected the tissue explants from OA patients undergoing total knee replacement and co-cultured them with mouse CAR-Macs targeting human FAP. In view of the obvious individual variation of SnCs in OA articular explants, we constructed DOX-induced senescent cartilage explants derived from undamaged cartilage tissue to avoid the bias (FIG. 12.1A). Increased percentages of FAP- and p21-positive cells confirmed a DOX-induced senescence phenotype of human cartilage explants. CAR-M co-culture eliminated FAP-positive senescent cells in senescent cartilage explants, decreased the expression of cartilage catabolic enzymes and promoted extracellular matrix synthesis (FIG. 12.1B, C). In addition, immunofluorescence staining proved that CAR-Macs infiltrated into cartilage explants and congregated around SnCs, while vector transduced Macs only remained on the surface of the explants (FIG. 12.2D). Although single explant examinations allowed for better control of experimental variables, they still cannot reflect critical crosstalk between synovium and cartilage in vivo. Therefore, we designed a cartilage-synovium co-culture model to test whether CAR-Macs could promote cartilage explants regeneration by eliminating SnCs in synovium: osteoarthritic synovial explants are first co-cultured with vector transduced Macs or CAR-Macs for 72 h, and then co-cultured with undamaged cartilage for 10 days (FIG. 12.1A). As expected, CAR-Macs can also effectively abrogate SnCs in OA-affected synovium (FIG. 12.2E, F). Interestingly, cartilage explants cultured in synovial condition medium (CM) co-cultured with CAR-M showed significantly fewer MMP13-positive cells and more proteoglycan synthesis than CM from osteoarthritic synovium alone (FIG. 12.3G, H). Taken together, these results demonstrated the senolytic activity of CAR-Macs in human joint explants and elucidated therapeutic effect of senescent cells removal in human OA model.

FIG. 13: Structure of the third-generation lentiviral vector clonetech pLVX-IRES-ZsGreen1 Vector.

### DETAILED DESCRIPTION

### Chimeric Antigen Receptor, CAR

CAR refers to a molecule that contains the fusion of extracellular antigen-binding domains, transmembrane domains, and molecules that fuse with intracellular signaling domains that activate or enhance the immune response. In some Embodiments, the extracellular antigen-binding domain of CAR contains scFv.

### scFv

scFv can be derived from the variable heavy and light regions of fused antibodies. scFv can be derived from Fab (rather than from antibodies, for example, obtained from Fab libraries). In some Embodiments, scFv fuses to the transmembrane domain and then to the intracellular signaling domain.

### CDR

CDR is defined as the complementary determining region of amino acid sequence of an antibody, which is the highly variable region of the heavy and light chains of immunoglobulins.

“Substantially identical” or “substantially homologous” means that a polypeptide or nucleic acid molecule exhibits at least approximately 50% homology or identical acid sequence with a reference amino acid sequence (e.g., any amino acid sequence described herein) or a reference nucleic acid sequence (e.g., any nucleic acid sequence described herein). In some Embodiments, the order is at least about 60%, at least about 65%, at least about 70%, at least about 75%, at least about 80%, at least about 85%. Sequence homologous or identical to at least 90%, at least 95%, at least 99%, or at least 100% of the amino acid or nucleic acid used for comparison.

### Macrophage

Macrophage is a kind of myeloid immune cell differentiated from monocytes after penetrate blood vessels, which are widely distributed in various organs of body tissues. Macrophage main physiological functions in normal tissues: mediating specific immune response by processing and presenting antigens; Phagocytosis and degradation of necrotic cells, debris and foreign bodies in the form of fixed cells or free cells, and then participate in non-specific reactions in the body; Secreting inflammatory cytokines activates lymphocytes or other immune cells that coordinate the inflammatory process.

### Senescent Cells And Senescence

The senscence mentioned in the invention generally refers to the senscence caused by the natural growth law of mammals or human or caused by certain diseases. One aspect of the senscent symptoms is due to the accumulation of senescent cells in the body. The accumulation of senescent cells due to disease may be caused by cancer, certain inflammatory diseases, or pain, or some physiological role in the occurrence and development of these diseases, such as the activation of certain proteins, certain genes, resulting in the production and accumulation of senescent cells, and thus the symptoms of senscence. These diseases include cardiac fibrosis, pulmonary fibrosis, atherosclerosis, Alzheimer's disease, diabetes, liver cirrhosis, chronic kidney disease, aging, osteoporosis or osteoarthritis, and the increase or accumulation of senescent cells in tumor subjects receiving senescence-inducing therapies, such as chemotherapy drugs. Accumulation can occur in diseased tissues or organs, or in systemic tissues or organs. In different tissues, the accumulation of senescent cells in organs leads to senescence, functional degeneration or normal physiological dysfunction of tissues and organs, which are caused by senescent cells.

### Surface Proteins of Senescent Cells

In general, senescent cells, compared with normal healthy non-senescent cells, always show an increase in the number of specific proteins or the appearance of neoproteins, which are present on the surface of the cell or expressed in the cytoplasm. Inhibiting or eliminating senescent cells is a means of treating senescent diseases. Antibodies or peptide fragments that inhibit the expression of senescent cell surface proteins or bind to senescent cell surface proteins are effective means. The invention discovers that fibroblast activation protein a (FAP, Fibroblast Activation Protein Alpha, Ensembl: ENSG00000078098, UniProtKB/Swiss-Prot: Q12884) is upregulated as a senescent cell surface protein in an in vitro and in vivo model of mammalian senescence.

### Vector

Expression of natural or synthetic nucleic acids is usually achieved by operationally attaching nucleic acids or parts thereof to promoters and incorporating the construction into expression vectors. The vector is usually able to replicate in mammalian cells and/or is also able to integrate into the mammalian cell genome. Typical vectors contain transcription and translation terminators, start sequences, and promoters that can be used to regulate the expression of desired nucleic acid sequences.

### Immune Cell

The immune cell in the invention has a CAR structure. The structure has extracellular regions, transmembrane regions and intracellular regions. Immune cells include phagocytes obtained from the subject, such as monocytes, macrophages, and/or dendritic cells. Non-restricted examples of subjects include humans, dogs, cats, mice, rats, and their genetically modified species. Preferably, the object is human. Cells can be obtained from many sources including peripheral blood monocytes, bone marrow, lymph nodes, spleen, umbilical cord, and tumor tissue.

### Therapy

The so-called therapy of the present invention is to eliminate, inhibit or delay the progression of a disease by administering reagents to an individual who has a disease, or applying drugs to an individual who needs to improve certain health conditions. On the one hand, the invention can provide any reagent, such as a reagent for binding the senescent cell surface protein FAP, such as antibodies, antibody fragments, peptide fragments, nucleic acids, chemical drugs that can bind FAP protein.

In another invention, a method that can utilize the modified engineered cells of the invention to treat or ameliorate the effects of age-related pathology in a subject in need, includes administering effective amounts of any of the engineered immune cells described herein to a subject in which the subject exhibits an increased accumulation of senescent cells compared to healthy control subjects.

In some embodiments, the age-related pathologies are ardiac fibrosis, pulmonary fibrosis, atherosclerosis, Alzheimer's disease, diabetes, liver cirrhosis, chronic kidney disease, aging, osteoporosis or osteoarthritis. In addition, or alternatively, in some Embodiments, senescent cells exhibit a senescence associated secretory phenotype (SASP). SASP may be caused by oncogenes (e.g., HRASG12D, NRASG12D; D38A, etc.) or induced by drugs (e.g., Cdk4/6 inhibitors (e.g., palbociclib), MEK inhibitors (e.g., trimetinib), doxorubicin). Of course, some of the engineered cells of the invention can also be applied to individuals to reduce senescent cells, cosmetic aspects, such as removing senescent cells from the skin to improve the skin, can also be used to reduce or remove senescent cells with FAP protein on the heart, liver.

### Experimental Examples

### Example 1: To Prove the Correlation Between FAP and Senescent Cells

FAP is Expressed in Senescent Cells:

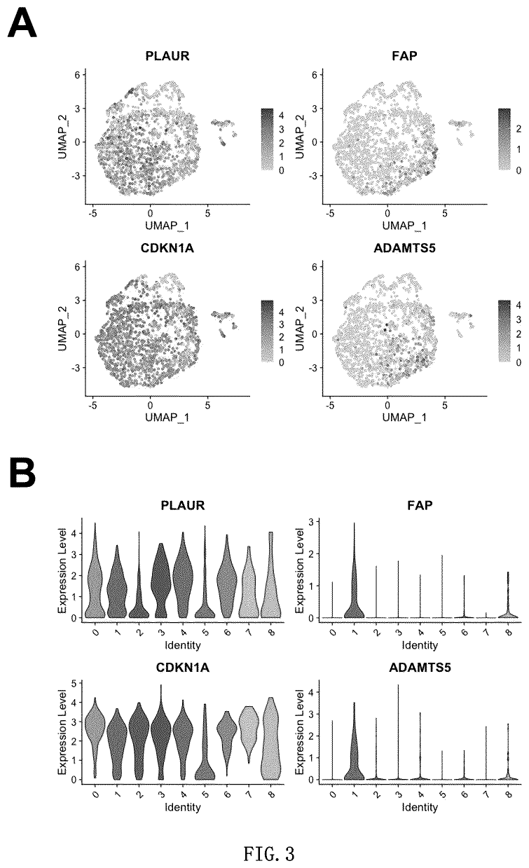

Identification of novel markers of senescent cells through single-cell data were analyzed single-cell RNA sequencing datasets (GSE104782) derived from articular cartilage of 10 OA patients. FIG. 3A shows UMAP dimensionality reduction in chondrocytes from OA patients, with significant heterogeneity in the expression of senescent cell signature genes and stroma-degrading genes. FIG. 3B shows in contrast to classical senescent cell markers CDKNIA and PLAUR, FAP is specifically expressed in senescent cell subsets that promote matrix degradation (identity=1).

FAP is Up-regulated in Senescent Cells:

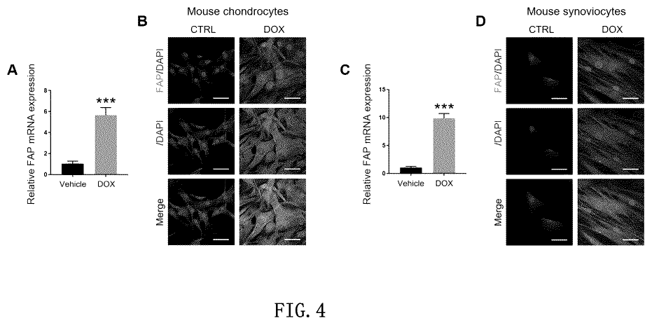

The primary mouse cells are treated with 500 ng/ml Doxorubicin (DOX) for 24 h and continued to be cultured with fresh complete medium for 7 days (the medium is changed every 2 days) to obtain senescent cells. The expression of FAP in senescent cells is detected using RT-qPCR and immunofluorescence staining (refer to Embodiment 9 for immunofluorescence staining). The result is shown in FIG. 4. FAP expression is significantly increased in primary mouse fibroblasts (FIG. 4C-D) and chondrocytes (FIG. 4A-B) after DOX treatment.

FAP is Expressed on the Surface of Senescent Cells:

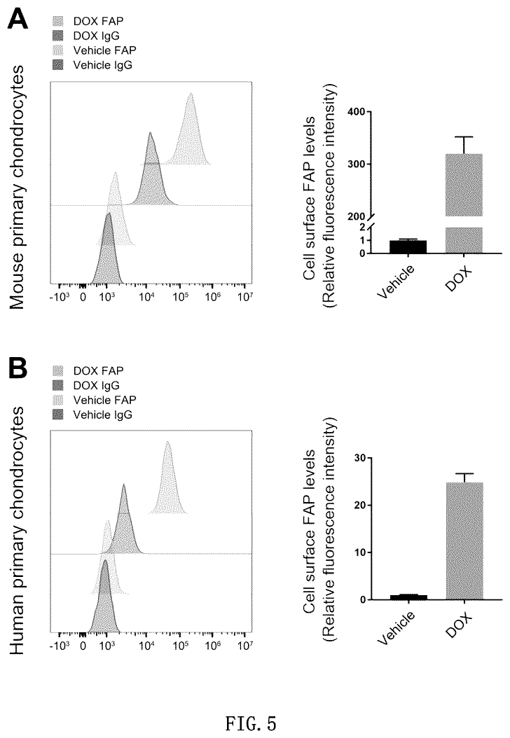

Chondrocytes obtained from mice and human are used for the experiment, and senescent cells are obtained as mentioned above. FAP expression levels are detected by flow cytometry. The adherent cells are digested with 0.2% EDTA-PBS buffer for 30 minutes to prevent damage to cell surface marker proteins, and the cell suspension is obtained by gently blowing the bottom of the culture dish with a pipette. Cell suspension is centrifuged at 200×g for 5 min, the cells are re-suspended with red fluorescentially labeled anti-FAP antibody (Abcam, ab207178) or isotype control IgG antibody at 1:200 dilution, and incubated at room temperature for 15-30 min. After incubation, the cell suspension is centrifuged to remove the supernatant and ished with PBS for 2-3 times. Finally, the cell density is adjusted to 1×10⁶/ml by resuspension cells with 200 μl PBS and the foil is used to avoid light. The cell suspension is transferred into the flow tube and tested on the flow cytometer. Flow cytometry analysis showed that FAP is highly expressed on the cell surface of mouse and human senescent chondrocytes. As shown in FIG. 5, FAP is highly expressed on the cell surface of mouse and human senescent chondrocytes (FIG. 5A-B).

FAP can be used as a senescent cell surface marker for OA and multi-organ senescence immunotherapy:

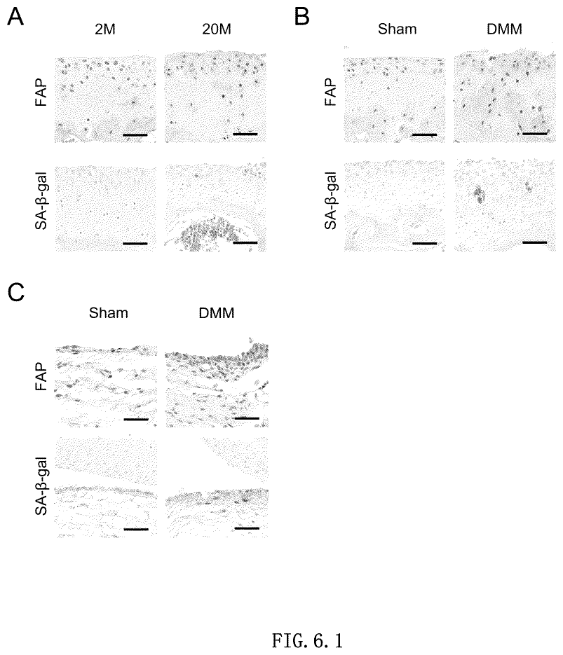

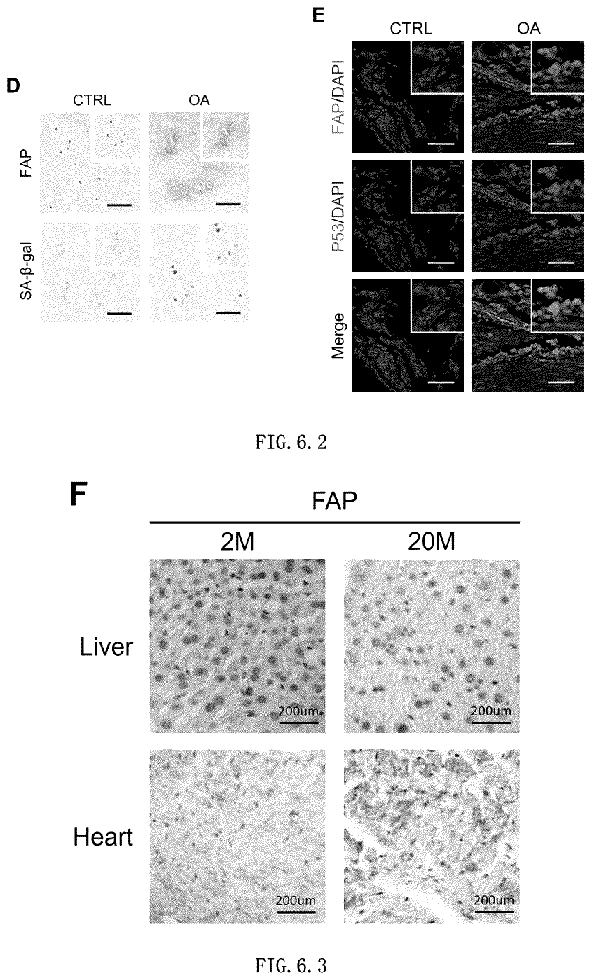

To verify the expression of FAP in aging and diseased tissues, joint, heart and liver tissues of young (2 months old) and old (20 months old) mice, joints with Destabilization of the Medial Meniscus (DMM)-induced osteoarthritis in mice, and cartilage and synovial tissues of patients with clinical osteoarthritis are obtained for tissue sections and FAP immunohistochemical staining. Aging markers such as SA-β-gal and p53 are detected by staining, the results are shown in FIG. 6.

FIG. 6 shows: the number of FAP-positive and senescence-associated β-galactosidase (SA-β-gal, a marker of aging) positive cells in mouse joint tissues (articular cartilage and synovial tissue) is significantly increased in both DMM-induced posttraumatic osteoarthritis (FIG. 6.1B-C) and age-related osteoarthritis model (FIG. 6.1A), but almost not detected in the sham joint. Similar to the results in mice, FAP is also upregulated in articular cartilage (FIG. 6.1D) and synovial tissue (FIG. 6.1E) of OA patients, while FAP expression is lower in normal joints. In addition, in both heart and liver tissues of mice, FAP are significantly increased in senescent individual (FIG. 6.2F). Together, these results demonstrate that FAP may be an ideal candidate target for the senolytic CAR strategy in immunotherapy for OA and multi-organ senescence.

### Example 2: Construction of Chimeric Antigen Receptor (CAR) Targeting FAP

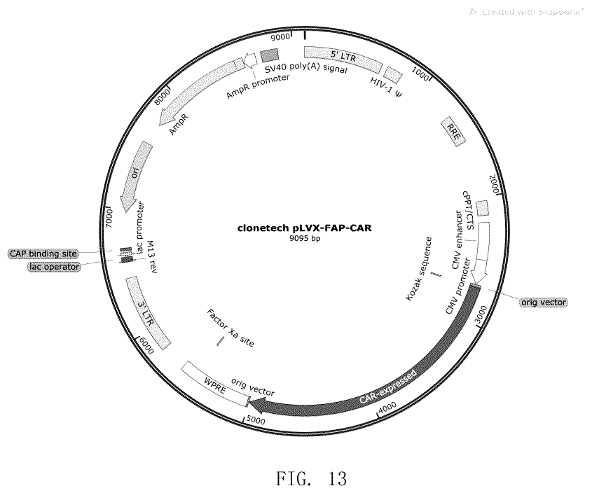

The entire sequence of the FAP-specific single chain antibody fragment, FAP-scFv (specific nucleic acid sequence such as SEQ ID: 12), CD8 transmembrane domain (specific sequence such as SEQ ID: 21), CD3ζ (specific sequence such as SEQ ID: 17), and CD28 intracellular domain (specific sequence such as SEQ ID: 15), is cut into the lentiviral vector plasmid clonetech pLVX-IRES-ZsGreen1 Vector (Plasmid structure shown in FIG. 13) by BsmBI and EcoRI enzymes, and the purified vector fragment and the CAR molecular DNA fragment are introduced into the downstream of the CMV promoter by T4 ligase to construct the recombinant plasmid.

The recombinant lentiviral vector plasmid includes CMV promoter, FAP single chain fragment variable (scFv), CD8 transmembrane domain, CD28 co-stimulatory domain, CD3 activation domain sequence.

The third-generation lentiviral plasmid (plasmid structure shown in FIG. 13) includes ampicillin resistance gene AmpR sequence, prokaryotic replicator pUC Ori sequence, PGK promoter, lentivirus 5 'LTR, lentivirus 3 'LTR, RRE cis-element, cPPT cis-element, and eWPRE enhanced posttranscriptional regulatory element of marmot hepatitis B virus.

### Eexample 3: Lentivirus Preparation

The lentiviral vector prepared in this Embodiment is the lentiviral vector prepared by the plasmid in Embodiment 2.

Preparation of target plasmid and packaging plasmid: The target plasmid (mFAP-CAR,GFP label; hFAP-CAR,GFP label; mCherry; luciferase, neomycin resistance) and the second-generation plasmids psPAX2 and pMD2.G are extracted with plasmid extraction kit (OMEGA). The concentration of corresponding plasmids is detected by Nano Drop (Thermo).

HEK293T cells (cat No.CL-0005, Procell Life Science & Technology Co., Ltd., Wuhan, China) for virus packaging: HEK293T cells are resuscitated and passaged in complete medium containing 1% antibiotics (Gibco)+10% FBS (Gibco)+high glucose DMEM (Gibco) for 2-3 generations, then digested with trypsin in the first 24 hours before virus packeting. 2.0×10⁶ cells are inoculated in a 10 cm cell culture dish and supplemented with DMEM complete medium to 10 ml. When the cell fusion reached 70-80%, the virus could be packeted after replacing 9 ml of new antibiotic-free DMEM complete medium.

Liposome transfection: 50 μl Lipofectamin 2000 (Invitrigen) is added into 500 μl Opti-MEM medium (Gibco) in 1.5 mL EP tube A, gently mixed, careful not to produce bubbles, let stand for 5 min at room temperature. Then, 8 μg target plasmid (prepared by the method of Embodiment 1), 6 μg psPAX2 plasmid, 6 μg pMD2.G plasmid and 500 μl Opti-MEM medium is added into another 1.5 mL EP tube B, gently mixed, careful not to produce bubbles, let stand for 5 min at room temperature. The above two kinds of mixed solution A and B are gently shaken and mixed, careful not to produce bubbles, let stand for 15 min at room temperature. The mixed is then dropped into the HEK293T with a pipette, and the dish is gently shaken and mixed, careful not to shake HEK293T cells off the bottom of the dish. Finally, the dish is placed in a 5% carbon dioxide incubator at 37° C., and the fresh DMEM complete medium is replaced after 12 h. Every 24 hours thereafter, the culture-medium containing the virus is changed and collected.

Virus harvesting (in the biosafety cabinet): after 48 h of culture, the supernatant of all HEK293T cells is transferred to a 50 mL centrifuge tube, and then centrifuged at 3000×g at 4° C. for 10 min. The supernatant is sucked out with a syringe and filtered by a 0.45 μm filter to obtain the FAP-CAR lentivirus, which is stored at −80° C. for later use.

### Example 4: Preparation of Chimeric Antigen Receptor Macrophages (CAR-M)

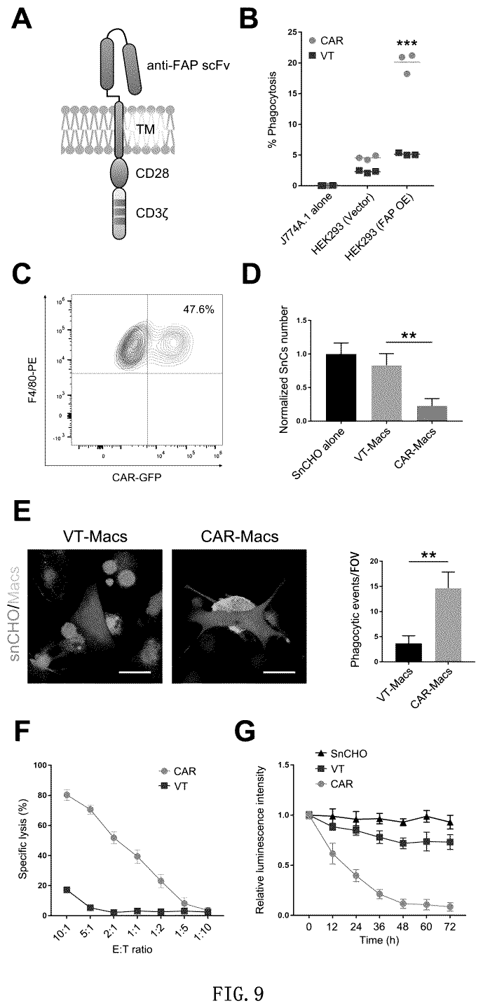

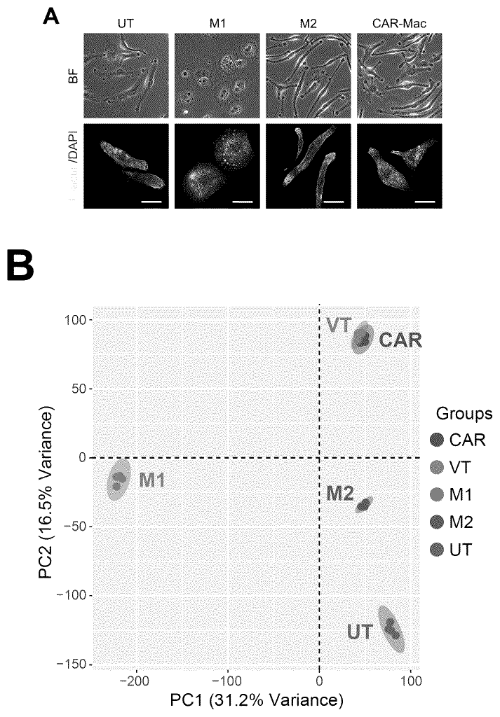

This Embodiment adopts the method of preparing chimeric antigen receptor macrophages (CAR-M) from mouse bone marrow derived macrophages (BMDM), and adopts the lentiviral vector prepared in Embodiment 3 during construction. The structure diagram of the expressed CAR is shown in FIG. 2, and the CAR expressed on the cell surface is shown in FIG. 9A. The details are as follows:

BMDM extraction:

C57BL/6N mice are killed by cervical dislocation, fully disinfected with 75% alcohol and effectively immobilized. The femur and tibia of the mice are isolated, be careful not to damage the bones, and then placed in a 10 cm cell culture dish containing 75% alcohol, transferred to a biosafety cabinet and further separated and removed the soft tissues. The isolated bones are ished with 1×PBS and transferred to another 10 cm cell culture dish containing low-glucose DMEM complete medium for further process.

The bone ends of the femur and tibia are removed with ophthalmic scissors, and the bone marrow cells are rinsed from a broken end of the bone with a 1ml low-sugar complete medium drawn with a 1 ml syringe into a 15 ml sterile centrifugation tube. This step is repeated 2-3 times until the bone marrow cavity became white. The collected cell suspension is centrifuged at 4° C., 200×g for 5 min, and the supernatant is discarded. 1 ml of red blood cell lysate is added, the lysate is left on ice for 5 min, then 5 ml of 1×PBS is added to terminate the lysate, centrifuged at 4° C., 200×g for 5 min, and the supernatant is discarded.

Cells are obtained by adding 5 mL DMEM complete medium and then filtered with a 70 μm cell sieve, centrifuged at 4° C., 200 ×g for 5 min, and discharging the supernatant.

Cell transfection:
1. The culture medium of BMDM in good growth state is sucked away, the cells are digested with Accutase, and the digested BMDM is seeded into the 6-well plate (Corning) according to 1.0×10⁶ cells/holes.
2. After cell adhesion, the cells are cultured with antibiotic-free DMEM complete medium.
3. Preparation of transfection working solution: Lentivirus prepared as in Embodiment 3 is used and Polybrene is added until the final concentration is 8 μg/ml. The transfection working solution is added into the 6-well plate at the rate of 2 ml/well, and the Blank group is added with the same amount of antibiotic-free DMEM complete culture medium containing 8 μg/ml Polybrene.
4. Seal the 6-well plate with a Parafilm and centrifuge at 37° C. and 800×g for 20 min.
5. After centrifugation, remove the viral medium, add 2 ml DMEM complete medium, and culture in 37° C., 5% carbon dioxide incubator overnight.
6. Repeat steps 2-4 if necessary.

CAR expression evaluation:

After the transfection cells are overgrown, Accutase is used to digest cells. CAR expression of the cells is detected by flow cytometry. The results are shown in FIG. 9C: FAP-specific CAR is successfully expressed on the surface of BMDM cells, which proves that the lentivirus spinfection could successfully introduce FAP-specific CAR into the mouse BMDM. Mouse BMDM transfected by spinfection could effectively express CAR.

### Example 5: Phagocytosis of CAR Macrophages (with FAP Antigen-binding Domain) on Senescent Cells

### 1. Phagocytosis Assay Based on Flow Cytometry

Viral vector construction and cell transfection refer to Embodiments 2, 3, and 4. 1×10⁶ GFP positive empty vector (empty vector in Embodiment 2) transfected macrophages (transfection method referred to Embodiment 3), CAR-M (CAR-M prepared in Embodiment 4) are co-cultured with medium alone (Macs alone), 5×10⁵ mCherry-positive HEK293T cells or FAP-overexpressing HEK293T cells at 37° C. for 24 h in 5% carbon dioxide incubator. The cells are digested with Accutase and fixed with 4% paraformaldehyde for 15 min. The percentage of mCherry positive cells in GFP positive cells is detected by flow cytometry. The result is shown in FIG. 9B. Flow cytometry analysis showed that FAP-specific CAR-M had a higher phagocytosis rate on FAP-positive target cells, while empty vector (containing GFP but without signal domains) transfected macrophages (VT-macs) do not have this selective phagocytosis (FIG. 9B).

### 2. Phagocytosis Assay Based on Microscopy

mCherry positive mouse primary chondrocytes are treated with 500 ng/ml doxorubicin for 24 h and cultured for 7 days to prepare mCherry positive senescent chondrocytes. The 2×10⁴ mCherry positive senescent chondrocytes are seeded into a 24-well plate with round coverslips (φ10 mm). After cell adhesion, 1×10⁵ GFP positive empty vector transfected macrophages and CAR-M are co-cultured with senescent chondrocytes at 37° C. and 5% carbon dioxide for 48 h. Glass coverslips are ished off with 1×PBS to remove unadherent cells, and then fixed with 4% paraformaldehyde for 15 min, DAPI is stained for 30 min. The cover slips are sealed with anti-fluorescence quenching tablet sealant. The cover slips are imaged using a confocal microscope (OLYMPUS) with 10× lens and mCherry and GFP channels. The average number of phagocytic events is calculated in 3 random fields per coverslip. Each experiment is repeated 3 times. The results are shown in FIG. 9E.

### 3. Phagocytosis Assay Based on High-content Cell Analyzer

2×10⁴ mCherry positive senescent chondrocytes are seeded into a 24-well plate with round coverslips (φ10 mm). After cell adhesion, 1×10⁵ GFP positive empty vector (empty vector in Embodiment 2) transfected macrophages (transfection method referred to Embodiment 3), CAR-M (CAR-M prepared in Embodiment 4) are co-cultured with senescent chondrocytes at 37° C. for 48 h in 5% carbon dioxide incubator. Glass coverslips are ished off with 1×PBS to remove unadherent cells, and then fixed with 4% paraformaldehyde for 15 min, DAPI is stained for 30 min. The coverslips are sealed with anti-fluorescence quenching tablet sealant. High-content cell analyzer (PerkinElmer) is used to measure the number of mCherry positive cells in 3 random fields in each coverslip, and each experiment is repeated 3 times. The results are shown in FIG. 9D.

Both FIGS. 9D and 9E show that CAR-Macs but not VT-Macscan selectively eliminate senescent chondrocytes. It is proved that CAR-M rather than empty vector transduced macrophages can phagocytic senescent chondrocytes.

### 4. Phagocytosis Assay Based on Live Cell Imaging

1×10⁵ mCherry positive senescent chondrocytes are seeded in 3.5 cm CELLview™ glass bottom dish (Greiner bio-one). After cell adhesion, 1×10⁵ GFP positive empty vector (empty vector in Embodiment 2) transfected macrophages (transfection method referred to Embodiment 3), CAR-M (CAR-M prepared in Embodiment 4) are co-cultured with senescent chondrocytes at 37° C. for 48 h in 5% carbon dioxide incubator. The cells are imaged with mCherry and GFP channels every 2-5 min using a confocal microscope (OLYMPUS) with a 10× lens for 24 h.

### 5. Cytotoxicity Assay

Macrophages transfected with GFP positive empty vector (empty vector in Embodiment 2) and CAR-M (CAR-M prepared in Embodiment 4) are used as effector cells and senescent chondrocytes expressing luciferase are used as target cells in luciferase based cell killing assay. 2×10⁴ target cells are seeded into an opaque 48-well plate with 3 multiples well in each group. After target cells adhesion, the effector cells are seeded according to the effector cells (E): target cells (T)=10:1-1:10, culture at 37° C. and 5% carbon dioxide for 48 h. The cells are ished 3 times with 1×PBS, the cells are lysed with lucifase lysate (Promega), centrifuge at 10000×g at 4° C. for 5 min, and 20 μl supercatant is added into an opaque 96-well plate, with 100 μl 150 μg/ml Xenolight D-luciferin, potassium salt (PerkinElmer, 122799) adding into each well and is incubated at 37° C. without light for 10 min. The fluorescence intensity is detected by Varioskan Flash microplate reader (Thermo), and the cell dissolution rate is calculated by the following formula: Specific lysis (%)=[1−(Sample signal−Background signal)/(Target alone signal−Background signal)]×100.

As shown in FIG. 9F-G, CAR-M rather than empty transfected macrophages showed dose- and time-dependent cytotoxicity to senescent chondrocytes. Compared with the control group, the CAR-M cells prepared by the invention can significantly kill senescent chondrocytes.

The results from this Embodiment suggest that FAP-specific CAR-M can effectively target senescent chondrocytes and mediate clearance of senescent cell activity.

### Example 6: Testing the Phagocytosis Efficiency Mediated by CAR Molecules With Different Intracellular Structures

In order to evaluate the phagocytosis efficiency of CAR-M with different co-stimulatory domains targeting senescent cells, CAR-M with different co-stimulatory domains were constructed, and the CAR-M of each group was co-cultured with DOX-treated primary human chondrocytes and fibroblasts (the treatment method was as in Embodiment 5), and the phagocytic effect of CAR-M was evaluated.

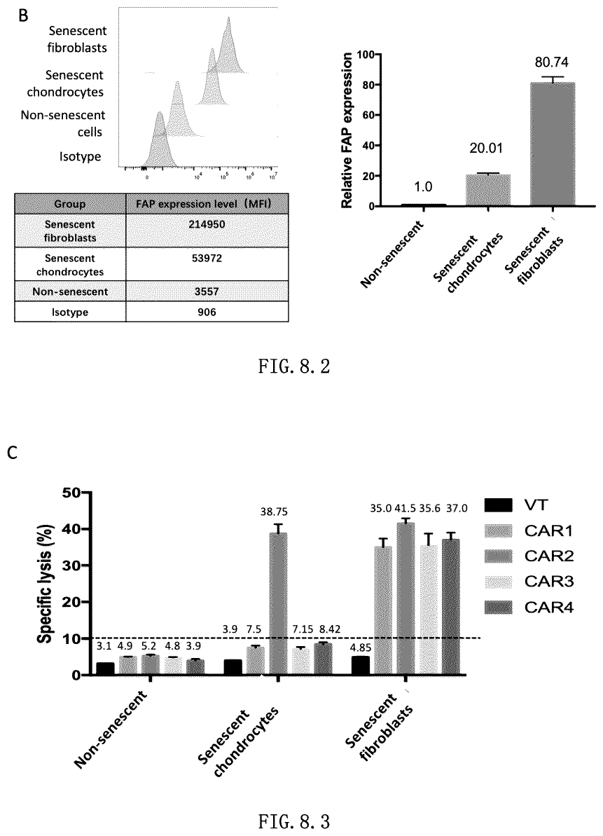

A total of 4 kinds of CAR are constructed, and the construction method is as in Embodiment 2. The construction schematic diagram is shown in FIG. 8.1A, including CAR-1, CAR-2, CAR-3, and CAR-4. They contain 4-1BB, CD28 intracellular domain, Dectin-1, and FcεRI common γ subunit in the co-stimulatory domain of the intracellular domain, and are consistent with each other in containing FAP specific scFv, CD8 hinge, CD8 transmembrane and CD3ζ intracellular region.

Refer to Embodiment 3 for the construction method of the lentiviral vectors of these four CARs, and the method of transfecting macrophages is the same as that of Embodiment 4.

Flow cytometry was used to detect the level of FAP protein on the cell surface. As shown in FIG. 8.2B, human senescent chondrocytes showed a lower level of upregulation of FAP protein (about 20 times) compared with non-senescent cells, while the FAP protein of human senescent fibroblasts showed a higher expression level (about 80 times).

After the CAR-M of each group was co-cultured with DOX-treated primary human chondrocytes and fibroblasts (the treatment method was as in Embodiment 5), the phagocytosis efficiency of CAR-M in each group was detected by flow cytometry, and the results are shown in FIG. 8.3C. The results of flow cytometry analysis showed that the four CAR-Ms had higher phagocytic activity on human senescent fibroblasts with higher FAP expression level, while for human senescent chondrocytes with lower FAP expression level, the CAR-M containing intracellular co-stimulatory fragment of CD28 had a higher phagocytosis rate on FAP-positive target cells (FIG. 8.3C). It shows that CAR-M containing CD28 intracellular co-stimulatory fragment has a lower response threshold to FAP on the surface of target cells.

### Example 7: Evaluation of the Polarization Phenotype of FAP-specific CAR-M

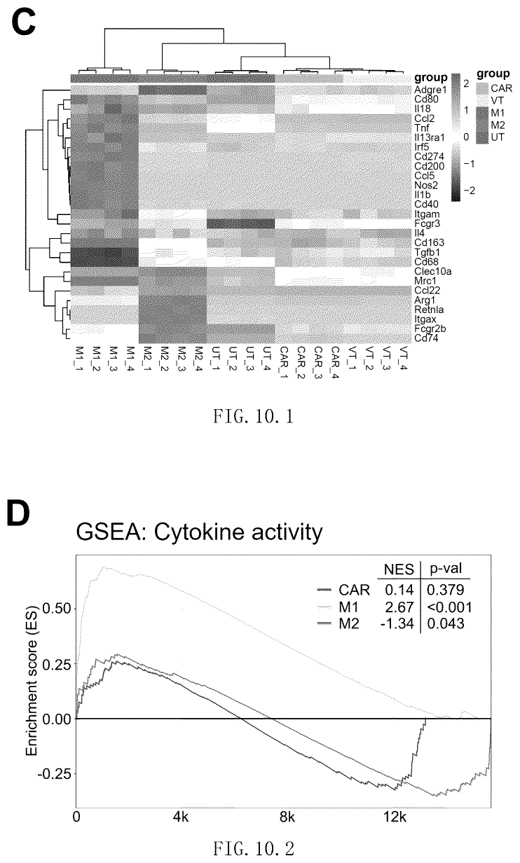

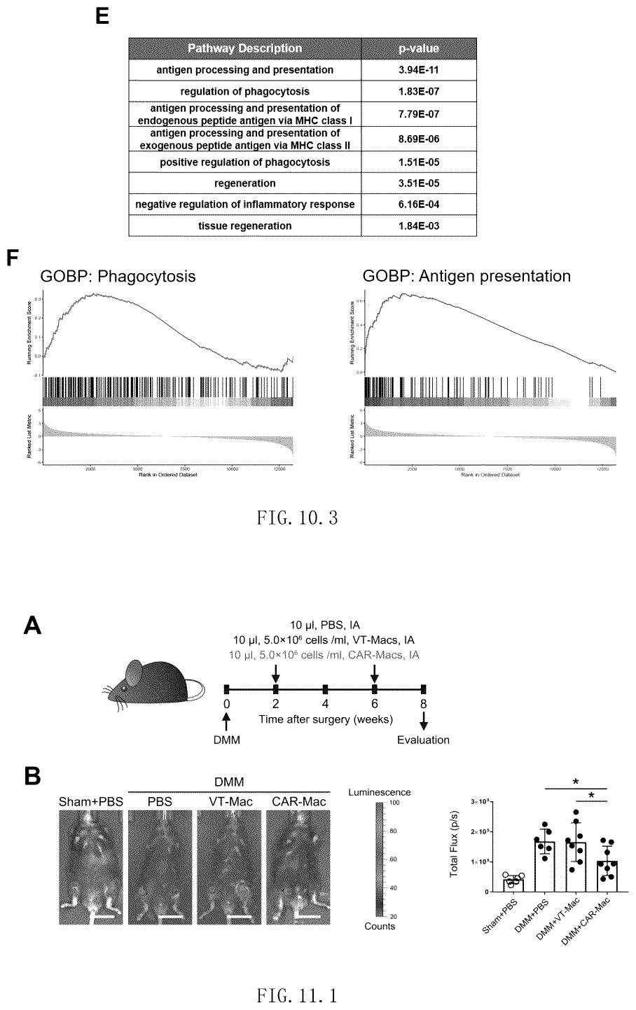

In order to evaluate the polarization phenotype of FAP-specific CAR-M, the CAR-M obtained in Embodiment 4 is used for experiments. Fluorescence microscopy is used to observe cell morphology by F-actin staining (Cytoskeleton, Inc. #PHDG1-A). Except for CAR-M (CAR-M prepared in Embodiment 4), compared with BMDM M0, M1 (BMDM incubated in 20 ng/ml IFN-γ and 100 ng/ml LPS for 48 h), M2 (BMDM incubated in 20 ng/ml IL-4 and 20 ng/ml IL-13) polarization, FAP-specific CAR-M showed an elongated shape with filamentous cytoskeleton staining, which is similar to the M2 (anti-inflammatory) phenotype (FIG. 10.1A). To further characterize the phenotype of FAP-specific CAR-M, bulk-RNA sequencing is performed. Here are the following groups: The CAR group is the CAR-transfected macrophages (prepared in Embodiment 4), the Empty group is the empty vector-transfected macrophages (prepared in Embodiment 5), the M1 group is the M1-type polarization, and the M2 group is the macrophages that alternatively activated M2 type polarization. The UTD group is the untreated BMDM. All of the above groups are treated by unbiased principal component analysis (PCA), CAR-M clustered toward alternatively activated M2 state and away from classically activated M1 (FIG. 10.1B). The unbiased hierarchical clustering heat map also shows that CAR-M is similar to M2 but significantly different from M1 (FIG .10.1C). Comparing the inflammation-related differences between CAR-M and activated macrophages, CAR-M did not show significant pro-inflammatory pathway enrichment compared to M1 polarizationmacrophages (FIG. 10.2D). In addition, CAR-M mediates phagocytosis and antigen presentation related path embodiments and key components (FIG. 10.3E, F). All these results suggest that FAP-specific CAR-M exhibits an anti-inflammatory like phenotype.

### Example 8: Human Explants Treated With CAR-M

Cartilage explants are obtained from 2 Kellgren-Lawrence (KL) grade 0 patients undergoing total knee replacement due to joint trauma, patients with a history of knee surgery or other joint disease (septic arthritis or tumor) are excluded, and the study obtained the informed consent of the patients and the approval of the institutional Ethics Committee.

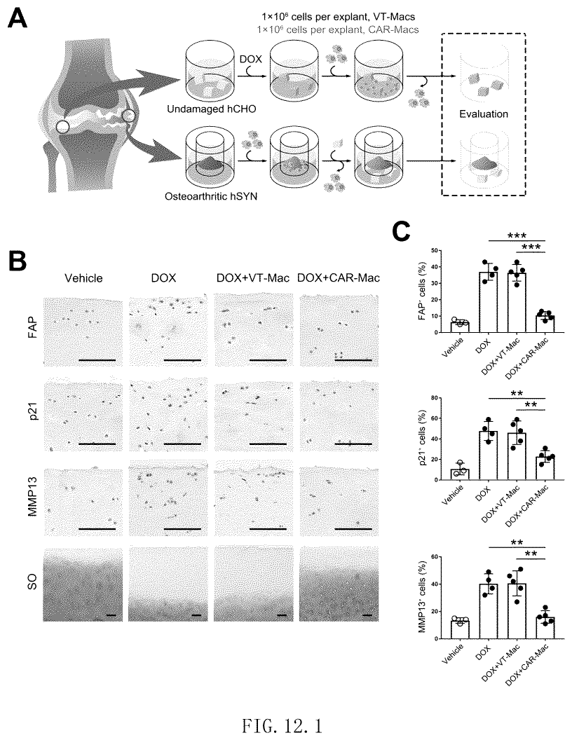

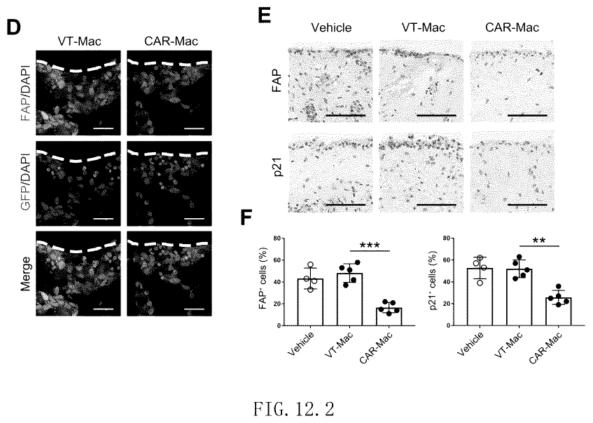

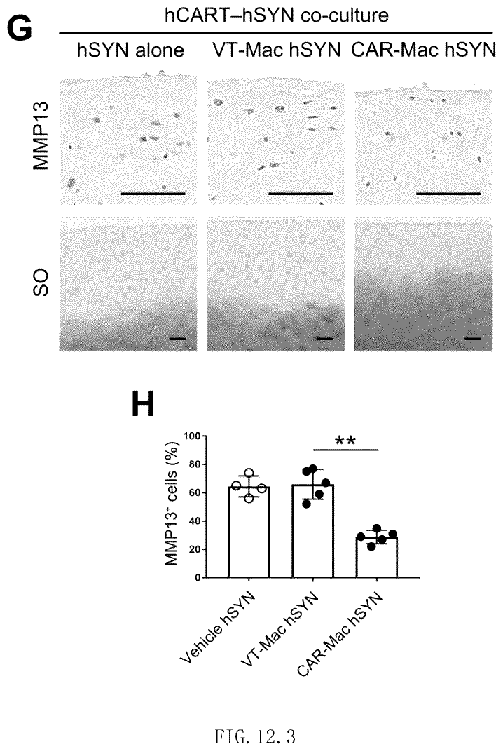

Given the significant individual variation in senescent cell numbers in OA joint explants, the invention constructed a DOX-induced senescent explant model to avoid bias (FIG. 12.1A). The whole layer of cartilage is cut off with a scalpel (#21 blade) and the sample is trimmed to about 5 mm×5 mm. The cartilage explant is placed in a 24-well plate with ultra-low attachment surface (Corning, USA). Add 1 ml DMEM/F12 complete medium containing 10% fetal bovine serum (FBS), 1% antibiotics, 1 mM sodium pyruvate, 50 μg/mL L-proline, 50 μg/mL L-ascorbate 2-phosphate and 1× insulin, transferrin, selenium solution (ITS-G). Pre-culture in 37° C., 5% carbon dioxide incubator for 72 h. The explants are then treated with DMEM/F12 complete medium containing 500 ng/ml doxorubicin for 48 h and continued to be cultured for 7 days to prepare the senescent human cartilage explants.

2×10⁵ GFP positive empty vector transfected macrophages (consistent with Embodiment 5), FAP-specific CAR-M (CAR-M prepared in Embodiment 4) are co-cultured with senescent cartilage explants at 37° C. for 72 h in 5% carbon dioxide incubator. The explants are ished in 1×PBS for 3 times, and fixed with 4% paraformaldehyde for 48 h away from light. Paraffin blocks are prepared by gradient dehydration, wax dipping and embedding of samples. The embedded paraffin blocks are sliced with a thickness of 5 μm. Immunofluorescence staining is performed on the sections.

To reflect the critical cross-talk between synovium and cartilage in OA pathology, the invention designed a cartilage-synovium co-culture model to test whether CAR-M can promote cartilage explants regeneration by eliminating senescent cells in synovium: Osteoarthritic synovium explants are first co-cultured with vector-transduced macrophages or CAR-M for 72 hours, and then with undamaged cartilage for another 10 days (FIG. 12.1A).

Immunofluorescence staining: The sections are ished in 1×PBS for 3 times, 5 min each time. The immune pen circles the sample area; Add 0.1% TritonX-100 to the sample for 15 min; The sections are ished in 1×PBS for 3 times, 5 min each time. Add 5% BSA to the sample for 1 h at room temperature; add 100 μl3% BSA diluted primary antibody to the sample: FAP (Abcam, ab207187) to ensure complete coverage of the sample, and incubate at 4° C. overnight; The sections are ished in 1×PBS for 3 times, 5 min each time. Add 100 μl3% BSA diluted corresponding fluorescent secondary antibody to the sample to ensure complete coverage of the sample, and incubate at room temperature for 1 h away from light; The sections are soaked in 1×PBS for 3 times and cleaned for 5 min each time. DAPI staining 30 min; The sections are ished in 1×PBS for 3 times, 5 min each time. The sections are sealed with anti-fluorescence quenching tablet sealant. The sections are imaged using a confocal microscope (OLYMPUS), 20× lens with mCherry and GFP channels. The result is shown in FIG. 12.

Immunohistochemical staining: DAB staining away from light (no more than 8 min). The staining is observed under microscope and terminated in time; Hematoxylin re-staining for 1 min; Rinse with running water for 1 min; 1% hydrochloric acid alcohol for color separation; bluing using tap-water for 5 min. The stained sections are dehydrated, transparent and sealed. The positive cells are photographed under an optical microscope and quantified.

The result is shown in FIG. 12:

FIGS. 12.1B and 12.1C show an increased percentage of FAP- and p21-positive cells confirming a DOX-induced senescent phenotype in human cartilage explants. CAR-M co-culture eliminate FAP-positive senescent cells from senescent cartilage explants, decreased the expression of cartilage catabolic enzymes and promoted the synthesis of extracellular matrix.

FIG. 12.2D shows immunofluorescence staining demonstrating that CAR-M infiltrate into cartilage explants and congregate around senescent cells, while vecor-transduced macrophages remained only on the surface of explants.

FIGS. 12.2E and F show that CAR-M can also effectively eliminate senescent cells in the osteoarthritic synovium. Cartilage explants cultured in synovial condition medium (CM) co-cultured with CAR-M shows fewer MMP13 positive cells and more proteoglycan synthesis than CM from osteoarthritic synovium alone (FIG. 12.3G, H).

Together, these results demonstrate the senolytic activity of CAR-Min human joint explants and elucidate the therapeutic role of senescent cells elimination in human OA models.

### Example 9: Mouse Osteoarthritis Treated With CAR-M

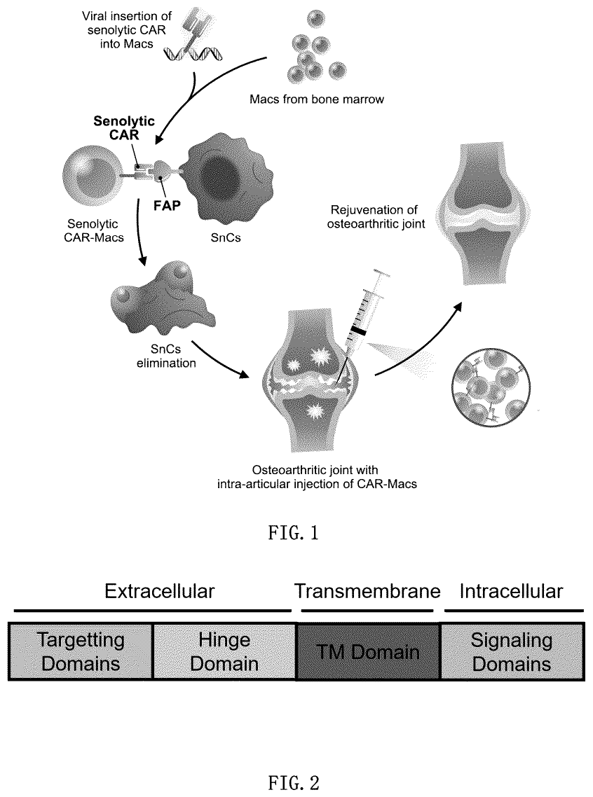

The CAR-M used in this embodiment is consistent with that prepared in Embodiment 4. The diagram of the treatment of in vivo osteoarthritis by CAR immune cells is shown in FIG. 1.

Mouse Destabilization of the Medial Meniscus (DMM) model:

The experimental mice are male p16-3MR mice aged 6-8 weeks (provided by Judith Campisi, Buck Institute for Research on Aging, USA). The mice are randomly divided into ABCD groups, in which group A is Sham group (n=5). DMM modeling is performed on the BCD group, n=8. The operation process is as follows:

Preoperative preparation: mice are anesthetized by intraperitoneal injection of 1% pentobarbital sodium (dose: 10 μl/g body weight). Mice are supine and fixed with the right hind limb at 90°, hair removal cream is used to remove the hair on the knee joint, the surgical field is exposed, and the skin is fully disinfected by 1% iodophor.

Microsurgical forceps and scissors are used to cut the skin length embodiments at the knee joint of the mice. The muscles and soft tissues are cut along the medial patellar ligament with a scalpel under the stereoscope (OlympusSZX7), and the patellar ligament is bluntly separated to expose the knee joint. The medial meniscus is connected to the tibial plateau via Medial Meniscus Tibial Ligament (MMTL).

The MMTL is cut off near the lateral side of the knee joint with microsurgical scissors and microsurgical knife (blade #11) to destabilize the medial meniscus, be careful not to scratch the articular cartilage (Sham group do not sever the ligament), and the wound is rinsed with sterile normal saline.

Joint cavity is sutured with 7-0 surgical sutures (VICRYL), muscle and skin are sutured with 6-0 surgical sutures (VICRYL), and skin is disinfected with 1% iodophor.

The mice are placed on the heating blanket until they woke up, and the mice had good mobility within 2 hours after surgery.

The CAR-M articular cavity injection:

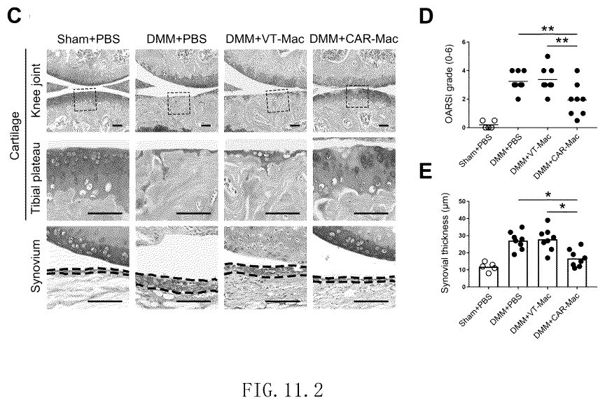

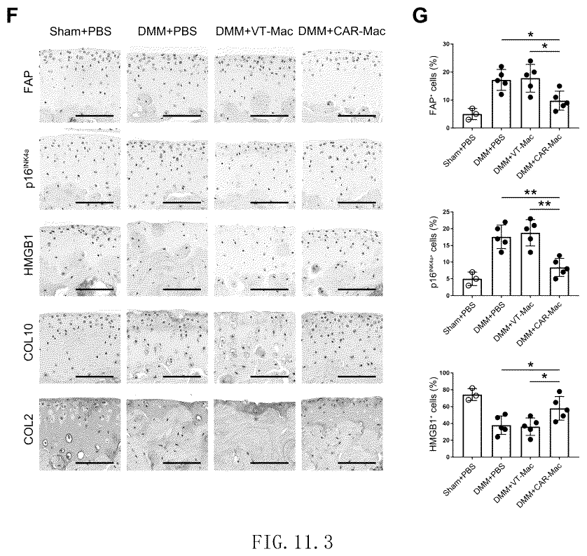

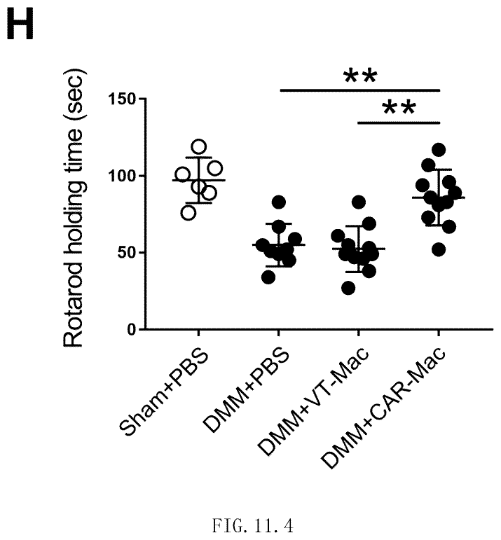

2 weeks after DMM surgery, intra-articular injection is performed using 25 μl microsyringe (Hamilton) with #33 needle (Hamilton). Group AB are injected with 10 μl vehicle solution (1×PBS), and group CD are injected with an equal amount of empty vector transdused macrophages (consistent with Embodiment 5) and CAR-M (CAR-M prepared in Embodiment 4), respectively, at a frequency of once a month, for a total of 2 doses, as shown in FIG. 11.1A.

Cell preparation: digesting and counting empty vector transdused macrophages (consistent with Embodiment 5) and CAR-M (CAR-M prepared in Embodiment 4), adjusting the cell density to 3.0×10⁶ cells/ml for standby use.

Preoperative preparation: refer to DMM model.

Intra-articular injection: The longitudinal skin incision is made in the middle of the knee joint, about 5 mm long, pull the skin to find the patellar ligament, the midpoint of the internal margin of the patellar ligament is taken as the injection point with a microsyringe, and the intra-articular (10 μl, 3.0×10⁶ cells/ml) is carried out at a depth of about 2 mm oblique above the back. After the injection is completed and stays for 5 s then pull out the needle.

Bioluminescence:

p16-3MR mice are anesthetized and 10 μl (150 μg/ml) XenolightRediJect Coelenterazine h (PerkinElmer, 760506) is injected into bilateral knee cavity. After 10 min, the fluorescence of senescent cells in the knee joint is quantified by IVIS Spectrum image system (PerkinElmer), Field of View: D, Medium Binning, Exposure Time: 5 min. The result is shown in FIG. 11. The fluorescence intensity of senescent cells in the CAR-M group is significantly lower than the empty vector transdused macrophages group. *p<0.05, **p<0.01, ***p<0.005; A/B; n=6, C/D; n=8.

Histology:

The fixed mouse joint samples are placed in decalcified solution (10% EDTA) for 3 weeks, the decalcified solution is replaced every 3 days. Paraffin blocks are prepared by gradient dehydration, wax dipping and embedding of samples. The embedded paraffin blocks are sliced with a thickness of 5 μm. The sections are dewaxed and stained with Safranin O &Fast Green and immunohistochemical staining.

Safranin O &Fast Green staining:

The dewaxed sections are first stained in 0.02% solid green solution for 5 min; Rinse with running water for 1 min; The sections are then stained in 0.1% Safranin O solution for 3 min; Rinse with running water for 1 min; Redye Fast Green for 5 min. The stained sections are dehydrated, transparent and sealed, photographed under the optical microscope and scored by OARSI. The result is shown in FIG. 11. Joint OARSI score and synovial thickness in CAR-M group are significantly lower than those in empty vector transdused macrophages group, *p<0.05, **p<0.01; Group A, n=5, group B/C/D, n=8.

Immunohistochemical staining:

The dewaxed sections are soaked in citric acid antigen repair solution and repaired overnight at 65° C. After antigen repairing, the sections are cooling to room temperature. The sections are ished in 1×PBS for 3 times, 5 min each time. The immune pen circles the sample area; Add 0.1% TritonX-100 to the sample for 15 min; The sections are ished in 1×PBS for 3 times, 5 min each time. Add 3% methanol hydrogen peroxide to the sample for 15 min; The sections are ished in 1×PBS for 3 times, 5 min each time. Adding 5% BSA to the sample for 1 h at room temperature; add 100 μl 3% BSA diluted primary antibody to the sample: FAP (Abcam, ab207187), p16INK4a (Abcam, ab241543), HMGB1 (Abcam, ab18256), COL10 (Invitrogen, 14-9771-82), COL2 (Santa Cruz, sc-52658) to ensure complete coverage of the sample, and incubate at 4° C. overnight; The sections are ished in 1×PBS for 3 times, 5 min each time. Add 100 μl 3% BSA diluted corresponding HRP-linked secondary antibody to the sample to ensure complete coverage of the sample, and incubate at room temperature for 1 h; The sections are ished in 1×PBS for 3 times, 5 min each time. DAB staining away from light (no more than 8 min). The staining is observed under microscope and terminated in time; Hematoxylin re-staining for 1 min; Rinse with running water for 1 min; 1% hydrochloric acid alcohol for color separation; bluing using tap-water for 5 min. The stained sections are dehydrated, transparent and sealed. The positive cells are photographed under an optical microscope and quantified.

The result is shown in FIG. 11:

CAR-M treatment significantly lowered the luminescence signal in the joint region of DMM mice, indicating effective senescent cells removal in the joint (FIG. 11.1B).

Histological assessment showed CAR-Macs significantly reduced cartilage degeneration and synovial inflammation in OA mice compared with vector-transduced Macs or PBS (FIG. 11.2C-E).

Immunostaining is performed for FAP, p16INK4a, and high-mobility-group box 1 (HMGB1) to identify senescent cells. In cartilage, the proportion of FAP- and p16INK4a-positive cells after DMM are significantly higher than those in the sham, while HMGB1 showed nuclear accumulation in the sham and loss after DMM. Notably, CAR-Macs not only significantly reduced the number of p16INK4a-positive cells and restored nuclear staining of HMGB1, but also reduced the number of collagen X-positive hypertrophic chondrocytes and increased the containing of collagen II in cartilage matrix in OA mice (FIG. 11.3F, G). The percentage of FAP- and p16INK4a-positive cells and the content of collagen X in CAR-M treatment group were significantly lower than those in empty vector transduced macrophage group, and the percentage of HMGB1 positive stained in nuclei cells and the content of collagen II in CAR-M treatment group were significantly higher than those in empty vector transduced macrophage group.*p<0.05, **p<0.01; Group A, n=3, group B/C/D, n=5. These results prove that CAR-M can be used as a therapeutic approach for post-traumatic OA and can treat age-related diseases.

Furthermore, Motor function assessed by Rotarod test revealed that CAR-Macs restored motor dysfunction associated with DMM surgery (FIG. 11.4H).

Collectively, these results suggested that FAP-specific CAR-M could be used as therapeutic agents in post-traumatic OA.

### Example 10: The Expression Efficiency of CAR in Primary Macrophages

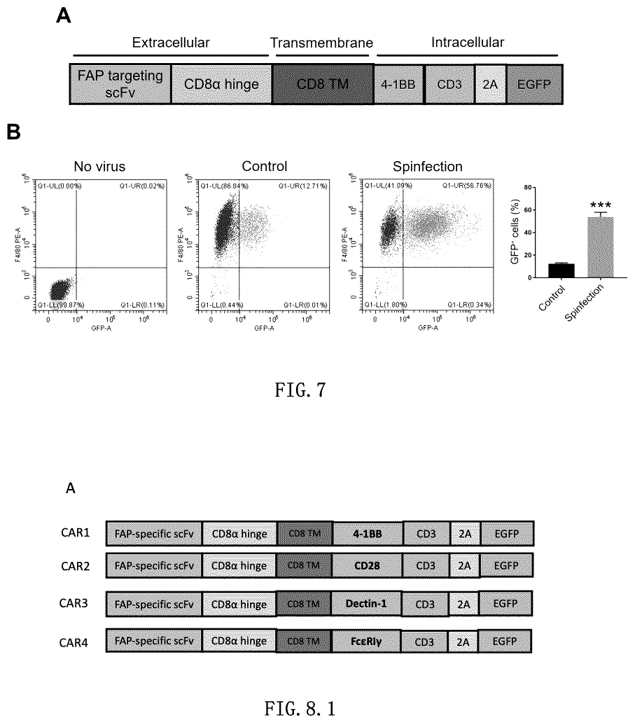

In this embodiment, the CAR structure diagram expressed by primary macrophages is shown in FIG. 7A. The extracellular domain is FAP-specific scFv (anti-mouse FAP (m.FAP) single chain fragment variable (scFV)) and the α hinge of CD8.

The transmembrane domain is CD8, the co-stimulatory domain is CD28, and the intracellular activation domain is CD35. The lentiviral vector FAP-CAR is generated in a method similar to Embodiment 2, and the CAR expression efficiency is detected by flow cytometry after transfecting primary mouse BMDMs in a method similar to Embodiment 3.

Primary mouse macrophages are isolated. After cell adhesion, the cells were washed once with antibiotic-free low-glucose DMEM complete medium.

Preparation of transfection working solution: Polybrene was added to the FAP-CAR lentivirus until the final concentration was 8 μg/ml.

The transfection working solution was added into the 6-well plate by 2 ml/well. Experiments were conducted in the following groups:

In the no virus group, transfection working solution was not added, and the same amount of antibiotic-free DMEM complete culture medium containing 8 μg/ml Polybrene was added.

The spinfection group was centrifuged at 800×g at 37° C. for 60 min and then recovered overnight in a 5% carbon dioxide incubator at 37° C.

The control group was cultured overnight in a 5% carbon dioxide incubator at 37° C.

After the transfected cells are overgrown, the cells are digested with Accutase, the expression of CAR of digested CAR-M is detected by flow cytometry. The results are shown in FIG. 7B, spinfection could significantly increase the expression efficiency of CAR up to 57%. The expression rate of CAR in primary macrophages was increased by 4.7 times by spinfection compared with the method without spinfection.

The following embodiments are also part of the invention:

Clause 1. A method for inhibiting or eliminating senescent cells, wherein the inhibition or removal of senescent cells by applying an agent that can inhibit or eliminate senescent cells to the individual. The agent that can inhibit or eliminate senescent cells includes agents targeting FAP proteins.

Clause 2. The method described in clause 1, wherein the FAP has an amino acid sequence as shown in SEQ ID: 1, and the nucleic acid encoding the FAP protein has a sequence as shown in SEQ ID: 2.

Clause 3. The method described in clause 2, wherein the reagent targeting FAP protein comprises a CAR extracellular FAP antigen-binding domain that can target the FAP antigen.

Clause 4. The method described in clause 3, wherein the reagents targeting FAP protein also include cells. The cells are engineered with a CAR targeting FAP protein. The cell type is one or more of monocytes, macrophages, or dendritic cells.

Clause 5. The method described in clause 4, wherein the cells are macrophages.

Clause 6. The method described in clause 1, wherein the senescent cells include any one or more of replication-induced, drug-induced and oncogene-induced senescent cells.

Clause 7. The method described in clause 6, wherein the drug-induced senescent cells include senescent cells induced by MEK inhibitors and/or Cdk4/6 inhibitors. The MEK inhibitors include trametinib and/or doxorubicin. The Cdk4/6 inhibitor includes palbociclib. The oncogenes include any one or more of HRASG12D, NRASG12D, and D38A.

Clause 8. A method for inhibiting or eliminating senescent cells, wherein the inhibition or removal of senescent cells by applying an agent that can inhibit or eliminate senescent cells to the individual. The agent that can inhibit or eliminate senescent cells includes agents expressing CAR that targeting FAP proteins.

Clause 9. The method described in clause 8, wherein the agent for inhibiting or eliminating senescent cells also includes cells. The cells are modified by CARs targeting FAP proteins. The cells include any one or more of monocytes, macrophages, or dendritic cells.

Clause 10. The method described in clause 8 or clause 9, wherein the diseases caused by senescent cells include any one or more of cardiac fibrosis, pulmonary fibrosis, atherosclerosis, Alzheimer's disease, diabetes, liver cirrhosis, chronic kidney disease, aging, osteoporosis or osteoarthritis.

Clause 11. A method for regulating the polarization of macrophages, wherein macrophages are transfected with lentiviral vectors to express CAR. The CAR contains a co-stimulatory domain of the CD28 intracellular domain that causes macrophages to produce anti-inflammatory M2-type polarization.

Clause 12. The method described in clause 11, wherein the co-stimulatory domain containing the CD28 intracellular domain is located in the intracellular domain of the CAR. The CAR also includes a targeted antigen-binding domain. The CD28 intracellular domain has an amino acid sequence as shown in SEQ ID: 14, or a sequence that is 60%, 70%, 80%, 85%, 90%, or more than 95% homologous to the amino acid sequence as shown in SEQ ID: 14. The CD28 intracellular domain has a nucleic acid sequence as shown in SEQ ID: 15, or a sequence that is 60%, 70%, 80%, 85%, 90%, or more than 95% homologous to a nucleic acid sequence as shown in SEQ ID: 15.

Clause 13. The method described in clause 12, wherein the targeted antigen-binding domain can be directional binding to target cells whose antigen density of the target protein reaches a response threshold, without binding to other cells.

Clause 14. The method described in clause 13, wherein the target protein includes, but not limited to, any of the following antigen components: PLAUR, GPNMB, mesothelin, CD1, CD1a, CD2, CD3, CD4, CD5, CD8, CD11A, CD14, CD15, CD16, CD18, CD19, C D21, CD20, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, C D79b, CD80, CD83, CD95, CD126, CD132, CD133, CD138, CD147, CD154, CDC27, GD2, CCL1 9, CCL21, CDK-4/m, CDKN2A, CTLA4, CXCR4, CXCR7, CXCL12, HER2, CAIX, CD171, LMP 1, EGFR, Muc1, GPC3, EphA2, EpCAM, MG7, CSR, ART-4.

All patents and publications referred to in the specification of the invention indicate that these are open techniques in the field and that the invention may be used. All patents and publications cited here are listed in the same references as each publication is specifically cited separately. The invention herein described may be realized in the absence of any one element or elements, a limitation or limitations, which are not specified herein. For example, in each instance here the term “contains”, “substantially consists of. . . . Composed of “and” composed of “Composition” can be replaced by the remaining two terms of either. The so-called “one” here means only the meaning of “one”, and does not exclude the inclusion of only one, but can also mean the inclusion of more than two. The terms and expressions used herein are not limited by them, nor is there any intention to indicate that the terms and interpretations described herein exclude any equivalent features, but it is understood that any suitable change or modification may be made within the scope of the invention and the claims. It is understood that the embodiments described in the invention are preferred embodiments, and that any ordinary person skilled in the art may make some changes and variations according to the contents described in the invention, which are also considered to be within the scope of the invention and the limits of independent claims and subsidiary claims.

1. A chimeric antigen receptor that can be expressed by cells comprising an extracellular domain, wherein the extracellular domain of the chimeric antigen receptor includes an antigen-binding domain that can bind a FAP protein.

2. The chimeric antigen receptor according to claim 1, wherein the antigen binding domain is a fragment of an antibody, wherein the FAP antigen has an amino acid sequence as shown in SEQ ID: 1, and the nucleic acid encoding the FAP antigen has a sequence as shown in SEQ ID: 2.

3. The chimeric antigen receptor according to claim 2, wherein the antibody fragment includes one or more of the single chain fragment variables (scFv), fragment of antigen binding (Fab), single chain Fab (scFab), or single chain immunoglobulin G (sclgG) antibody fragments.

4. The chimeric antigen receptor according to claim 3, wherein the antibody fragment comprises a heavy chain and/or a light chain, the heavy chain includes any one or more of VHCDR1, VHCDR2, VHCDR3, the light chain includes any one or more of VLCDR1, VLCDR2, VLCDR3, the VHCDR1, VHCDR2, VHCDR3, VLCDR1, VLCDR2, and VLCDR3 have amino acid sequences as shown in SEQ ID: 4-9, respectively, or a sequence that is 60%, 70%, 80%, 85%, 90%, or more than 95% homologous to any one or more of the amino acid sequences shown in SEQ ID: 4˜9.

5. The chimeric antigen receptor, as described in claim 4, wherein the antibody fragment is scFv, the scFv contains a heavy chain and/or a light chain, and wherein the heavy chain has an amino acid sequence as shown in SEQ ID: 10, the light chain has an amino acid sequence as shown in SEQ ID: 11, or a sequence that is 60%, 70%, 80%, 85%, 90%, or more than 95% homologous to the amino acid sequence shown in SEQ ID: 10 and/or SEQ ID: 11.

6. The chimeric antigen receptor according to claim 5, wherein the scFv having a nucleotide sequence encoding the scFas indicated by SEQ ID: 12 and/or SEQ ID: 13, or a sequence that is 60%, 70%, 80%, 85%, 90%, or more than 95% homologous to a nucleotide sequence as indicated by SEQ ID: 12 and/or SEQ ID: 13.

7. The chimeric antigen receptor according to claim 1, wherein the chimeric antigen receptor further comprises an intracellular domain and a transmembrane domain, and the intracellular domain includes a co-stimulatory domain and an intracellular activation domain.

8. The chimeric antigen receptor according to claim 7, wherein the co-stimulatory domain is a fragment comprising one or more of the intracellular domains selected from 4-1BB (CD137), CD80, CD86, FcsRIγorCD28, and wherein the FcsRIγ has an amino acid sequence as shown in SEQ ID: 22, or a sequence that is 60%, 70%, 80%, 85%, 90%, or more than 95% homologous to the amino acid sequence as shown in SEQ ID: 22; or the FcsRIγ has a nucleic acid sequence encoding the FcsRIγ as shown in SEQ ID: 23, or a sequence that is 60%, 70%, 80%, 85%, 90%, or more than 95% homologous to a nucleic acid sequence as shown in SEQ ID: 23.

9. The chimeric antigen receptor according to claim 8, wherein the co-stimulatory domain is a CD28 intracellular domain that has an amino acid sequence as shown in SEQ ID: 14, or a sequence that is 60%, 70%, 80%, 85%, 90%, or more than 95% homologous to the amino acid sequence as shown in SEQ ID: 14, or the CD28 intracellular domain has a nucleic acid sequence as shown in SEQ ID: 15, or a sequence that is 60%, 70%, 80%, 85%, 90%, or more than 95% homologous to a nucleic acid sequence as shown in SEQ ID: 15.

10. The chimeric antigen receptor according to claim 7, wherein the intracellular activation region is FcγRI or CD3ζ.

11. The chimeric antigen receptor according to claim 10, wherein the intracellular active region is CD3ζ and wherein the CD3ζ has an amino acid sequence as shown in SEQ ID: 16, or a sequence that is 60%, 70%, 80%, 85%, 90%, or more than 95% homologous to the amino acid sequence as shown in SEQ ID: 16, or the CD3ζ has a nucleic acid sequence as shown in SEQ ID: 17, or a sequence that is 60%, 70%, 80%, 85%, 90%, or more than 95% homologous to a nucleic acid sequence as shown in SEQ ID: 17.

12. The chimeric antigen receptor according to claim 7, wherein the transmembrane domain contains CD28 or CD8 and wherein the CD28 and CD8 have an amino acid sequence as shown in SEQ ID: 18˜19, or a sequence that is 60%, 70%, 80%, 85%, 90%, or more than 95% homologous to the amino acid sequence as shown in SEQ ID: 18˜19; and the CD28 and CD8 have nucleic acid sequences as shown in SEQ ID: 20˜21 or sequences that are 60%, 70%, 80%, 85%, 90%, or more than 95% homologous to nucleic acid sequences as shown in SEQ ID: 20˜21.

13. The chimeric antigen receptor according to claim 1, wherein the extracellular domain of the chimeric antigen receptor also includes a spacer region connecting the targeted FAP antigen binding domain and the transmembrane domain.

14. The chimeric antigen receptor according to claim 13, wherein the spacer region includes a part of CD28, a part of CD8, an immunoglobulin CH2CH3 region, a part of CD3, and any one or more of the hinge regions of IgGl, the CD28 and CD8 have an amino acid sequence as shown in SEQ ID: 18˜19, respectively, or a sequence that is 60%, 70%, 80%, 85%, 90%, or more than 95% homologous to the amino acid sequence as shown in SEQ ID: 18˜19, the CD28 and CD8 have nucleic acid sequences as shown in SEQ ID: 20˜21, respectively, or sequences that are 60%, 70%, 80%, 85%, 90%, or more than 95% homologous to nucleic acid sequences as shown in SEQ ID: 20˜21.

15. The chimeric antigen receptor according to claim 1, wherein the cells comprise monocytes, macrophages, or dendritic cells.

16. The chimeric antigen receptor according to claim 15, wherein the cell expresses the chimeric antigen receptor by transfecting the cell with a vector, and the vector is a plasmid vector, a viral vector, a retrotransposon, a site-insertion vector, or a suicide expression vector.

17. The chimeric antigen receptor according to claim 16, wherein the vector is a lentiviral vector.

18. The chimeric antigen receptor according to claim 15, wherein the cells are macrophages.

19. A method for preparing modified cells containing chimeric antigen receptors, wherein the macrophages are transfected with lentivirus as a carrier to express the chimeric antigen receptor, wherein the extracellular domain of the chimeric antigen receptor includes antigen-binding domain that can bind an FAP, the transfection is performed by spinfection.

20. A method for inhibiting or eliminating senescent cells comprising applying an agent to the individual, wherein the agent includes cells that comprises a chimeric antigen receptor including an extracellular domain, wherein the extracellular domain of the chimeric antigen receptor includes an antigen-binding domain that can bind a FAP protein.


SEQUENCE LISTING (24 sequences)

SEQ ID NO: 1 - protein, 735 aa, synthetic construct
MKTWVKIVFGVATSAVLALLVMCIVLRPSRVHNSEENTMRALTLKDILNGTFSYKTFFPNWISGQEYLHQSADNNIVLYNIETGQSYTILSNRTMLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 2 - DNA, 2621 bp, synthetic construct
  source 1..2621: mol_type = other DNA
gtcctggcttcagcttccaactacaaagacagacttggtccttttcaacggttttcacagatccagtgacccacgctctgaagacagaattagctaactttcaaaaacatctggaaaaatgaagacttgggtaaaaatcgtatttggagttgccacctctgctgtgcttgccttattggtgatgtgcattgtcttacgcccttcaagagttcataactctgaagaaaatacaatgagagcactcacactgaaggatattttaaatggaacattttcttataaaacattttttccaaactggatttcaggacaagaatatcttcatcaatctgcagataacaatatagtactttataatattgaaacaggacaatcatataccattttgagtaatagaaccatgctttggagatactcttacacagcaacatattacatctatgaccttagcaatggagaatttgtaagaggaaatgagcttcctcgtccaattcagtatttatgctggtcgcctgttgggagtaaattagcatatgtctatcaaaacaatatctatttgaaacaaagaccaggagatccaccttttcaaataacatttaatggaagagaaaataaaatatttaatggaatcccagactgggtttatgaagaggaaatgcttgctacaaaatatgctctctggtggtctcctaatggaaaatttttggcatatgcggaatttaatgatacggatataccagttattgcctattcctattatggcgatgaacaatatcctagaacaataaatattccatacccaaaggctggagctaagaatcccgttgttcggatatttattatcgataccacttaccctgcgtatgtaggtccccaggaagtgcctgttccagcaatgatagcctcaagtgattattatttcagttggctcacgtgggttactgatgaacgagtatgtttgcagtggctaaaaagagtccagaatgtttcggtcctgtctatatgtgacttcagggaagactggcagacatgggattgtccaaagacccaggagcatatagaagaaagcagaactggatgggctggtggattctttgtttcaacaccagttttcagctatgatgccatttcgtactacaaaatatttagtgacaaggatggctacaaacatattcactatatcaaagacactgtggaaaatgctattcaaattacaagtggcaagtgggaggccataaatatattcagagtaacacaggattcactgttttattctagcaatgaatttgaagaataccctggaagaagaaacatctacagaattagcattggaagctatcctccaagcaagaagtgtgttacttgccatctaaggaaagaaaggtgccaatattacacagcaagtttcagcgactacgccaagtactatgcacttgtctgctacggcccaggcatccccatttccacccttcatgatggacgcactgatcaagaaattaaaatcctggaagaaaacaaggaattggaaaatgctttgaaaaatatccagctgcctaaagaggaaattaagaaacttgaagtagatgaaattactttatggtacaagatgattcttcctcctcaatttgacagatcaaagaagtatcccttgctaattcaagtgtatggtggtccctgcagtcagagtgtaaggtctgtatttgctgttaattggatatcttatcttgcaagtaaggaagggatggtcattgccttggtggatggtcgaggaacagctttccaaggtgacaaactcctctatgcagtgtatcgaaagctgggtgtttatgaagttgaagaccagattacagctgtcagaaaattcatagaaatgggtttcattgatgaaaaaagaatagccatatggggctggtcctatggaggatacgtttcatcactggcccttgcatctggaactggtcttttcaaatgtggtatagcagtggctccagtctccagctgggaatattacgcgtctgtctacacagagagattcatgggtctcccaacaaaggatgataatcttgagcactataagaattcaactgtgatggcaagagcagaatatttcagaaatgtagactatcttctcatccacggaacagcagatgataatgtgcactttcaaaactcagcacagattgctaaagctctggttaatgcacaagtggatttccaggcaatgtggtactctgaccagaaccacggcttatccggcctgtccacgaaccacttatacacccacatgacccacttcctaaagcagtgtttctctttgtcagactaaaaacgatgcagatgcaagcctgtatcagaatctgaaaaccttatataaacccctcagacagtttgcttattttattttttatgttgtaaaatgctagtataaacaaacaaattaatgttgttctaaaggctgttaaaaaaaagatgaggactcagaagttcaagctaaatattgtttacattttctggtactctgtgaaagaagagaaaagggagtcatgcattttgctttggacacagtgttttatcacctgttcatttgaagaaaaataataaagtcagaagttcaagtgcta

SEQ ID NO: 3 - DNA, 108 bp, synthetic construct
  source 1..108: mol_type = other DNA
aaacggggcagaaagaagctcctgtatatattcaaacaaccatttatgagaccagtacaaactactcaagaggaagatggctgtagctgccgatttccagaagaagaa

SEQ ID NO: 4 - protein, 5 aa, synthetic construct
SYGMS

SEQ ID NO: 5 - protein, 17 aa, synthetic construct
TTNNNGGVTYYPDSVKG

SEQ ID NO: 6 - protein, 8 aa, synthetic construct
YGYYAMDY

SEQ ID NO: 7 - protein, 16 aa, synthetic construct
RSSQSIVHSNGNTYLE

SEQ ID NO: 8 - protein, 7 aa, synthetic construct
KVSNRFS

SEQ ID NO: 9 - protein, 9 aa, synthetic construct
FGGSHVPYT

SEQ ID NO: 10 - protein, 116 aa, synthetic construct
QVQLKESGGLVQPGGSLKLSCAASGFTFSSYGMSWVRQTADKRLELVATTNNNGGVTYYPDSVKGRFTISRDNAKNTLYLQMSSLQSEDTAMYYCARYGYYAMDYWGQGISVTVSS

SEQ ID NO: 11 - protein, 112 aa, synthetic construct
DVLMTQTPLWLPVSLGDQASISCRSSQSIVHSNGNTYLEWYLQKPGQSPKLLIYKVSNRFSGVPDRFSGSGSGTDFTVKISRVEAEDLGVYYCFGGSHVPYTFGGGTKLEIK

SEQ ID NO: 12 - DNA, 729 bp, synthetic construct
  source 1..729: mol_type = other DNA
gatgtgctgatgacccagacacccctgtggctgcctgtgagcctgggggatcaggccagcatctcctgcaggagcagccagagcatcgtgcactccaacgggaacacatacctggagtggtacctgcagaagcccggccagagccccaagctgctgatctacaaggtgagcaaccggttcagcggcgtgcccgacaggttcagcgggagcggctccgggacagacttcaccgtgaagatcagccgggtggaggccgaggacctgggggtctactactgcttcgggggctcccacgtcccctacaccttcggggggggcaccaagctggagatcaagggcggcgggggctccgggggcggggggtccgggggcggcgggtcccaggtgcagctgaaggagagcggcgggctggtgcagcccggcgggtccctcaagctgagctgcgccgccagcgggttcacctttagcagctacggcatgagctgggtgcggcagaccgccgacaagcggctggagctggtggccacaaccaacaacaacgggggcgtgacatactaccccgatagcgtgaagggcaggtttaccatctccagggataacgccaagaacacactgtacctgcagatgagcagcctgcagagcgaggacacagccatgtactactgcgcccggtacgggtactacgccatggactactggggccagggcattagcgtgacagtgagcagc

SEQ ID NO: 13 - DNA, 729 bp, synthetic construct
  source 1..729: mol_type = other DNA
caggtgcagctgaaggagagcgggggcctggtgcagcccggcgggagcctgaagctgagctgcgccgccagcggcttcaccttcagcagctacgggatgagctgggtgcggcagaccgccgataagaggctggagctggtcgccaccaccaacaacaacgggggggtgacatactacccagatagcgtgaagggccggttcacaatcagcagggataacgccaagaacacactgtacctgcagatgtccagcctgcagagcgaggacaccgccatgtactactgcgcccggtacggctactacgccatggactactggggccagggcatcagcgtgacagtgtcctccggcggcggcggcagcggggggggcggcagcggggggggcgggtccgatgtgctgatgacacagacacccctgtggctgccagtgagcctgggcgaccaggccagcatctcctgcaggagcagccagagcatcgtgcactccaacgggaacacatacctggagtggtacctgcagaagcctgggcagagccccaagctgctgatctacaaggtgtccaacaggttcagcggcgtgcccgatcggttcagcgggagcgggagcggcaccgacttcacagtgaagatcagcagggtggaggccgaggacctgggcgtgtactactgcttcggggggagccacgtgccctacacattcgggggcggcacaaagctggagatcaag

SEQ ID NO: 14 - protein, 41 aa, synthetic construct
RSKRSRLLHSDYMNMTPRRPGPTRKHYQPYAPPRDFAAYRS

SEQ ID NO: 15 - DNA, 123 bp, synthetic construct
  source 1..123: mol_type = other DNA
agatcaaagcgctccaggttgcttcatagcgactacatgaatatgacacctcggcgccccgggcccacacgaaagcactaccagccctacgccccgccaagagacttcgctgcttatcgcagc

SEQ ID NO: 16 - protein, 112 aa, synthetic construct
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 337 bp, synthetic construct
  source 1..337: mol_type = other DNA
agagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcg

SEQ ID NO: 18 - protein, 214 aa, synthetic construct
MLRLLLALNLFPSIQVTGNKILVKQSPMLVAYDNALSCKYSYNLFSREFRASLHKGLDSAVEVCWYGNYSQQLQVYSKTGFNCDGKLGNESVTFYLQNLYQTDIYFCKIEVMYPPPYLDNEKSNGTIIHVKGKHLCPSPLFPGPSKPFWVLVWGGVLACYSLLVTVAFIIFWVRSKRSRLLHSDYMNMTPRRPGPTRKHYQPYAPPRDFAAYRS

SEQ ID NO: 19 - protein, 71 aa, synthetic construct
PTTTPAPRPPTPAPTIASQPLSLRPEACRPAAGGAVHTRGLDFACDIYIWAPLAGTCGVLLLSLVITLYCN

SEQ ID NO: 20 - DNA, 321 bp, synthetic construct
  source 1..321: mol_type = other DNA
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 21 - DNA, 213 bp, synthetic construct
  source 1..213: mol_type = other DNA
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaac

SEQ ID NO: 22 - protein, 42 aa, synthetic construct
RLKIQVRKAAIASREKADAVYTGLNTRSQETYETLKHEKPPQ

SEQ ID NO: 23 - DNA, 126 bp, synthetic construct
  source 1..126: mol_type = other DNA
cgactgaagatccaagtgcgaaaggcagctataaccagctatgagaaatcagatggtgtttacacgggcctgagcaccaggaaccaggagacttacgagactctgaagcatgagaaaccaccacag

SEQ ID NO: 24 - protein, 36 aa, synthetic construct
KRGRKKLLYIFKQPFMRPVQTTQEEDGCSCRFPEEE